(19) United States
(12) Patent Application Publication
(10) Pub. No.: US 2012/0157512 A1
(43) Pub. Date: Jun. 21, 2012

(54) Preventing and Curing Beneficial Insect Diseases Via Plant Transcribed Molecules

(75) Inventors: Eyal Ben-Chanoch, Miami, FL (US); Nitzan Paldi, Moshav Bar-Giori (IL); Gal Yarden, Doar-Na Negev (IL)

(73) Assignee: MONSANTO TECHNOLOGY LLC, St. Louis, MO (US)

(21) Appl. No.: 13/391,103

(22) PCT Filed: Aug. 22, 2010
(86) PCT No.: PCT/IB10/53776

§ 371 Date: Feb. 17, 2012

Related U.S. Application Data
(60) Provisional application No. 61/235,707, filed on Aug. 21, 2009.

(51) Int. Cl. A61K 31/7088; C12N 5/10; C12N 1/21; A01H 5/00; A61K 31/713; A61P 31/14; A61P 31/04; A61P 31/12; A61P 33/02; A61P 31/10; A61P 33/00; C12N 15/82; A61P 31/00

(52) U.S. Cl. 514/44 A; 435/320.1; 435/419; 435/252.2; 800/298; 800/285; 800/286

(57) ABSTRACT

Methods and compositions for transforming plants to express polynucleotides capable of gene silencing gene expression in pathogens of beneficial insets such as IAPV, Nosema species and Varroa mites, and methods for using the transgenic plants for reducing infection and susceptibility of bees to Colony Collapse Disorder are provided.

### FIELD AND BACKGROUND OF THE INVENTION

The invention relates to compositions and methods for reducing susceptibility to pathogenic organisms in beneficial insects using RNA interference technology, and more particularly, to the use of plants expressing dsRNA for prevention and treatment of infections in honeybees.

Beneficial Insects and Insect Pathogens

The importance of honeybees and other pollinating insects to the global world economy far surpasses their contribution in terms of honey production. The United States Department of Agriculture (USDA) estimates that every third bite we consume in our diet is dependent on a honeybee to pollinate that food. The total contribution of pollination in terms of added value to fruit crops exceeds $15 billion per annum, with indirect potential consequence of $75 billion dollars.

The unprecedented influence of human society, namely, monoculture and technology, overuse of pesticides, and global warming with subsequent emerging diseases, are all leading to what experts are referring to as the sixth major extinction of species on Earth. This in turn has led to a catastrophic decline in the population of beneficial insects, with pollinating insects such as bees most severely affected. Indeed, in the U.S. there has been a massive impact on agriculture and a threat to the human food chain, in direct consequence of what is known as Colony Collapse Disorder (CCD) of honeybees, which is characterized by the rapid loss from a colony of its adult bee population. Decline in honeybee colonies worldwide has reached 25-30% or more loss per season since 2006, a significant portion of which is due to Colony Collapse Disorder (CCD).

Beneficial insects, such as honeybees or bumblebees used for pollination, as well as antagonists of pests used in biocontrol programs, such as parasitic wasps, all have a range of pests and pathogens that cause them an array of diseases. Such diseases reduce their fecundity and thus reduce their capacity as beneficial insects. For honeybees these include numerous parasites and pathogens, including viruses, bacteria, protozoa, Endoparasites and mites, each with characteristic modes of transmission.

CCD has been associated with the Israeli Acute Paralysis Virus (IAPV). Weakened colonies are also more susceptible to opportunistic infection associated with other pathogens such as viruses [e.g. deformed wing virus (DWV), Kashmir bee virus (KBV), acute bee paralysis virus (ABPV) and black queen cell virus (BQCV)] bacteria (e.g. Melissococcus pluton, European foulbrood) and microsporidia (e.g. Nosema). In addition, infestation of pests such as the parasitic Varroa mites weakens the bees and breaches the protective insect exoskeleton, making them vulnerable to further infection by pathogenic species.

Chemical means for controlling these infections have been employed, such as Fumagillin for Nosema infection and pyrethroid and organophosphate miticides for Varroa. They have not only failed to eradicate the pathogens and pests, but also pose a serious risk of toxicity and contamination of the bees' honey.

Methods for Silencing Using siRNAs/dsRNA

RNA interference (dsRNA and siRNA) has been shown effective in silencing gene expression in a broad variety of species, including plants, with wide ranging implications for cancer, inherited disease, infectious disease in plants and animals. It was also shown in a variety of organisms that dsRNA or their siRNA derivatives can be used to arrest, retard or even prevent a variety of pathogens, most notably viral diseases (see, for example, WO/2003/004649 to Tenllado et al).

It has been shown in some species that RNAi mediated interference spreads from the initial site of dsRNA delivery, producing interference phenotypes throughout the injected animal. Recently the same spreading effect of dsRNA has been demonstrated in bee larva, as well as detection of SID transmembrane channels considered responsible for endocytic uptake and spreading effect of dsRNA in humans, mouse and C. elegans (Aronstein et al, J. Apic Res and Bee World, 2006; 45:20-24).

Application of RNA interference technology for insects that are plant pests and other plant pests has been suggested. Moderate RNAi-type silencing of insect genes by feeding has been demonstrated (Turner et al., Insect Mol Biol 2006; 15:383; and Araujo et al., Insect Mol. Biol 2006; 36:683). dsRNA absorbance via honey has also been demonstrated (Aronstein et al., J Apiculture Res Bee World 2006; 45:20-24).

U.S. Pat. No. 6,326,193 refers to the use of recombinant insect viruses such as baculoviruses expressing dsRNA to silence selected insect genes for pest control. PCT application WO 99/32619 describes the use of dsRNA for reducing crop destruction by plant pathogens or pests such as arachnids, insects, nematodes, protozoans, bacteria, or fungi. PCT patent application WO 2004/005485 describes RNAi sequences and transgenic plants designed to control plant-parasitic nematodes.

US Patent Application 20030154508 describes pest control with a dsRNA against a cation-amino acid transporter/channel protein. PCT patent application WO 02/14472 describes an inverted repeat and a sense or antisense nucleic acids for inhibiting target gene expression in a sucking insect. US patent application 20030150017 describes the use of RNA molecules homologous or complementary to a nucleotide sequence of a plant pest such as nematodes and insects.

Raemakers et al (PCT Applications WO 2007/080127 and WO 2007/080126) have disclosed transgenic plants expressing RNAi for controlling pest infestation by insects, nematodes, fungus and other plant pests. Among the sequences taught are sequences targeting essential genes of insects, including the honeybee. Waterhouse et al (US Patent Application 2006 0272049) and Van De Craen (US Patent Application No. 2010068172) also disclosed transgenic plants expressing dsRNA directed to essential genes of plant insect pests, for use as pesticides and insecticides. Boukharov et al. (US Patent Application 2007 0250947) disclosed dsRNA in transgenic plants for targeting plant parasitic nematodes. While expression and processing of dsRNA were demonstrated, no actual inhibition of infestation with the dsRNA was shown.

Recently, Paldi et al demonstrated that ingestion by bees of a segment of IAPV-dsRNA leads to viral RNA degradation and is successful in silencing IAPV infection and preventing bee mortality (PCT Application WO2009/060429), and that Nosema-specific dsRNA can specifically and differentially silence ADP/ATP transporter gene expression (Paldi et al., Applied Environ Microbiol, Epub Jul. 9, 2010).

Although direct application of dsRNA to the commercially grown honeybees can be facilitated by providing it in bee feed as described by Paldi et al. (PCT Application WO2009/060429), there is no way to protect feral population of bees that are still the mainstay of pollination of crops in most of the world. Thus, there is a need for improved methods of providing the beneficial insects with protection from pathogenic disease agents.

### SUMMARY OF THE INVENTION

According to an aspect of some embodiments of the present invention there is provided a nucleic acid construct comprising a polynucleotide expressing a nucleic acid agent downregulating expression of a gene product of an insect pathogen, the polynucleotide operably linked to a cis-regulatory element operable in plants.

According to an aspect of some embodiments of the present invention there is provided a cell comprising a nucleic acid construct comprising a polynucleotide expressing a nucleic acid agent downregulating expression of a gene product of an insect pathogen, the polynucleotide operably linked to a cis-regulatory element operable in plants.

According to some embodiments of the invention, the cell is a plant cell.

According to some embodiments of the invention, the cell is an Agrobacterium.

According to an aspect of some embodiments of the present invention there is provided a plant expressing a nucleic acid construct comprising a polynucleotide expressing a nucleic acid agent downregulating expression of a gene product of an insect pathogen, the polynucleotide operably linked to a cis-regulatory element operable in plants.

According to some embodiments of the invention, the plant is a flowering plant selected from the group consisting of rapeseed, alfalfa and clover.

According to some embodiments of the invention, the plant is expressing the nucleic acid construct in floral nectary tissues, extrafloral nectarines or pollen.

According to an aspect of some embodiments of the present invention there is provided a method of reducing the susceptibility of a insect to a disease caused by an insect pathogen, the method comprising providing to the insects plants expressing a nucleic acid construct a nucleic acid construct comprising a polynucleotide expressing a nucleic acid agent downregulating expression of a gene product of an insect pathogen, the polynucleotide operably linked to a cis-regulatory element operable in plants, such that the insect feeds from the plants and ingests the nucleic acid agent, thereby reducing the susceptibility of the insect to the pathogen.

According to some embodiments of the invention the insect is a pollinating insect or a herbivorous insect.

According to some embodiments of the invention the insect is a bee and the plants express a nucleic acid agent complementary to a nucleic acid sequence selected from the group consisting of SEQ ID NOs: 60-252698, 252699-256728, and 256782-266260.

According to some embodiments of the invention the insect is a bee and the plants express a nucleic acid agent complementary to a nucleic acid sequence selected from the group consisting of SEQ ID NOs: 55-59, 253194, 254090, 259289, 256752, 256761 and 256762.

According to an aspect of some embodiments of the present invention there is provided a method for reducing the susceptibility of an bee to a disease caused by an insect pathogen comprising providing the bee with a plant expressing a nucleic acid construct comprising a polynucleotide expressing a nucleic acid agent down-regulating expression of a gene product of a IAPV, Nosema and/or Varroa bee pathogen, the polynucleotide operably linked to a cis-regulatory element operable in plants, such that the bee feeds from the plants and ingests the nucleic acid agent, thereby reducing the susceptibility of the bee to the pathogen.

According to an aspect of some embodiments of the present invention there is provided a method of reducing the susceptibility of honeybees to Colony Collapse Disorder (CCD), the method comprising providing to the honeybees plants expressing a nucleic acid construct comprising a polynucleotide expressing a nucleic acid agent downregulating expression of a gene product of a IAPV, Nosema and/or Varroa bee pathogen, the polynucleotide operably linked to a cis-regulatory element operable in plants, such that the bee feeds from the plants and ingests the nucleic acid agent, thereby reducing the susceptibility of the bee to the pathogen.

According to an aspect of some embodiments of the present invention there is provided a method of producing a plant capable of reducing the susceptibility of an insect feeding from the plant, plant tissues or plant secretions to a disease caused by an insect pathogen, the method comprising transforming cells of the plant with a nucleic acid agent downregulating expression of a gene product of a IAPV, Nosema and/or Varroa bee pathogen, the polynucleotide operably linked to a cis-regulatory element operable in plants, selecting transformed plants or plant cells expressing insect pathogen-specific dsRNA, and propagating the plants or plant cells to produce a plant or plant cells capable of reducing the susceptibility of an insect feeding from the plant.

According to some embodiments of the invention, the nucleic acid agent is selected from the group consisting of a dsRNA, an antisense RNA and a ribozyme.

According to some embodiments of the invention the dsRNA is selected from the group consisting of siRNA, shRNA and miRNA.

According to some embodiments of the invention the cis regulatory element is active in floral nectary tissues, extrafloral nectarines and/or pollen.

According to some embodiments of the invention the nucleic acid agent is greater than 15 base pairs in length.

According to some embodiments of the invention the nucleic acid agent is 19 to 25 base pairs in length.

According to some embodiments of the invention the nucleic acid agent is greater than 30 base pairs in length.

According to some embodiments of the invention the insect is a bee.

According to some embodiments of the invention the insect pathogen is selected from the group consisting of a virus, a bacteria, a parasitic protozoan, a fungus a nematode and a mite.

According to some embodiments of the invention the insect pathogen is a virus.

According to some embodiments of the invention the insect is a bee and the virus is Israel Acute Paralysis Virus.

According to some embodiments of the invention the nucleic acid agent is complementary to a viral nucleic acid sequence as set forth in any of SEQ ID NOs: 256752, 256761, 256762 and 311552.

According to some embodiments of the invention the nucleic acid agent is complementary to a viral nucleic acid sequence as set forth in any of SEQ ID NOs: SEQ ID NOs: 256782-266260.

According to some embodiments of the invention the nucleic acid sequence is as set forth in SEQ ID NO: 311533.

According to some embodiments of the invention the insect is a bee and the pathogen is a Nosema parasite.

According to some embodiments of the invention the nucleic acid agent is complementary to a Nosema nucleic acid sequence as set forth in any of SEQ ID NOs: 55-252698.

According to some embodiments of the invention the Nosema parasite is N. cerana and the gene product is an mRNA encoding a Nosema ATP/ADP transporter protein or homologue thereof.

According to some embodiments of the invention the ATP/ADP transporter protein or homologue thereof is selected from the group consisting of proteins encoded by SEQ ID NOs: 44, 45, 46 and 47.

According to some embodiments of the invention the insect is a bee and the pathogen is a Varroa destructor mite.

According to some embodiments of the invention the gene product is an Varroa specific mRNA encoding a polypeptide selected from the group consisting of NADH dehydrogenase subunit 2, ATP synthetase subunit 8, ATP synthetase subunit 6, sodium channel and cytochrome oxydase subunit I.

According to some embodiments of the invention the insect is a bee and the nucleic acid construct comprises at least two nucleic acid agents, each of the nucleic acid agents downregulating expression of at least one gene product of at least one bee pathogen.

According to some embodiments of the invention the insect is a bee and the at least one bee pathogen is selected from the group consisting of a virus, a bacteria, a parasitic protozoan, a fungus, a nematode and a mite.

According to some embodiments of the invention the at least one bee pathogen comprises at least two bee pathogens.

According to some embodiments of the invention the nucleic acid agent corresponds to conserved sequences in the at least two bee pathogens.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic map of the plasmids pJETCPset1 and pJETCPset2, bearing the insert encoding the IAPV-specific dsRNA and its inverted repeat, respectively;

FIG. 2 is a schematic map of the plasmid pRNA69CPset1;

FIG. 3 is a schematic map showing transcription of the IAPV-specific dsRNA from pRNA69 CP-IR, using T7 primers;

FIG. 4 is a gel analysis of transcription products from pRNA69 CP-IR showing correctly sized, dsRNA products. Lane 1 is the molecular weight size ladder. Lane 2—endogenous bee RNA (single stranded)(completely degraded)+RNAse A. Lane 3—transcription product of pRNA IR-CP#1, no RNAse A. Lane 4—dsRNA transcription product of pRNA IR-CP#1+RNAse A. Lane 5—dsRNA transcription product of pRNA IR-CP#2+RNAse A. Lane 6—endogenous bee RNA (single stranded)+DICER (resistant). Lane 7—transcription product of pRNA IR-CP#1, no DICER. Lane 8—dsRNA transcription product of pRNA IR-CP#1+DICER. Lane 5—dsRNA transcription product of pRNA IR-CP#2+DICER. Note the correct size of the transcription products, their resistance to single strand-specific RNAse I (lanes 4 and 5), and complete degradation with double strand-specific DICER (lanes 8 and 9);

FIG. 5 is a schematic showing pRNA69 CP-IR and the plasmids pBART and pART27;

FIG. 6 is a schematic of pBART and pART27 comprising the IAPV-specific dsRNA expression cassettes;

FIG. 7 is a gel analysis of transcription products from pBART IR-CP#1 and IR-CP#2, showing correctly sized, dsRNA products. Lane 1—endogenous bee RNA from a bee fed Nosema-specific dsRNA (mixed ss and ds RNA)+RNAse A (note the partial degradation and single resistant 760 bp fragment). Lane 2—transcription product of pRNA69 IR-CP#1+RNAse A (resistant). Lane 3—dsRNA transcription product of pBART IR-CP#1, no RNAse A. Lane 4—dsRNA transcription product of pBART IR-CP#1+RNAse A. Lane 5—dsRNA transcription product of pBART IR-CP#2+RNAse A. Lane 6—dsRNA transcription product of pART27 IR-CP#1+RNAse A. Lane 7—dsRNA transcription product of pART27 IR-CP#1+RNAse A. Lane 8—dsRNA transcription product of pART27 IR-CP#1, no RNAse A. Lane 9—Molecular weight ladder. Lane 10—endogenous bee RNA from a bee fed Nosema-specific dsRNA (mixed ss and ds RNA)+DICER (note the complete degradation of the ds RNA fragment, and resistance of the ss ribosomal and m-RNA). Lane 11—transcription product of pRNA69 IR-CP#1+DICER (degraded). Lane 12—dsRNA transcription product of pBART IR-CP#1, no DICER. Lane 13—dsRNA transcription product of pBART IR-CP#1+DICER. Lane 14—dsRNA transcription product of pBART IR-CP#2+DICER. Lane 15—dsRNA transcription product of pART27 IR-CP#1+DICER. Lane 16—dsRNA transcription product of pART27 IR-CP#1+DICER. Lane 17—dsRNA transcription product of pART27 IR-CP#1, no RNAse A. Note the correct size of the transcription products, their resistance to single strand-specific RNAse I (lanes 3-7), and complete degradation with double strand-specific DICER (lanes 13-17).

### DESCRIPTION OF SPECIFIC EMBODIMENTS OF THE INVENTION

The present invention, in some embodiments thereof, relates to plant-expressed compositions and methods for reducing susceptibility to pathogenic organisms in beneficial insects using RNA interference technology, and more particularly, but not exclusively, to the use of plants expressing bee pathogen-specific dsRNA for prevention and treatment of infections in honeybees.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Pathogens causing disease of beneficial insects, such as bees and other pollinators, inflict untold economic damage on modern agriculture, and threaten to alter traditional patterns of pollination and gene flow critical to modern agriculture. Effective and economically feasible means for reducing occurrence and severity of insect-pathogen infection, and the efficient delivery of these means to the affected insects are much needed.

Paldi et al have demonstrated gene silencing of viral and microsporidian bee pathogens (IAPV-PCT Application WO2009/060429; and Nosema—Paldi et al., Applied Environ Microbiol, Epub Jul. 9, 2010), and reduced susceptibility to infection in bees fed with pathogen-specific dsRNA. However, provision of pathogen-specific dsRNA to the critically important feral bee populations, which are not concentrated in easily located hives, and to bees foraging during seasons of plentiful flower forage, requires both increased quantities of the pathogen-specific dsRNA, and improved methods for its dispersion and delivery to the insects.

Bees and other beneficial, herbivorous insects feed off plant tissues, such as nectar, pollen and soft parts of the plant. The present inventors have surprisingly found that providing plants expressing insect pathogen-specific dsRNA in plant tissues and secretions available to the insects in the vicinity of bees and other beneficial insects can be effective in reducing disease and infection caused by these pathogens in the beneficial insects. Bees and other beneficial insects ingesting the plants or plant tissues benefit from gene silencing of the pathogenic organisms, and reduced susceptibility to the associated diseases and infections. In particular embodiments, the present invention provides methods and compositions for transforming plants to express polynucleotides capable of gene silencing of IAPV, Nosema species and Varroa mites, and methods for using the transgenic plants for reducing infection and susceptibility of bees to Colony Collapse Disorder.

Thus, according to one aspect of the present invention, there is provided a method of reducing the susceptibility of an insect to a disease caused by an insect pathogen, the method comprising providing to the insect plants expressing a nucleic acid construct comprising a polynucleotide expressing a nucleic acid agent down-regulating expression of a gene product of an insect pathogen, said polynucleotide operably linked to a cis-regulatory element operable in plants, such that said insect feeds from said plants and ingests said nucleic acid agent, thereby reducing the susceptibility of said insect to said pathogen.

As used herein, the term “beneficial insect” is defined as any of several members of the class Insecta, including Apterygota (wingless) and Pterygota (winged), which perform a function desirable by humans, such as insects which assist production in agriculture, and contribute to pest control, habitat integration, aesthetics and ‘natural vitality’ in horticulture and gardening. In some embodiments of the present invention, the beneficial insects are herbivorous or pollinating insects. A non-limiting list of beneficial insects, including herbivorous and pollinating species, includes bees, the minute pirate bug (Orius), the big-eyed bug (Geocoris), the soldier beetles (Antharidae), the green lacewings (Chrysophidae) and hoverflies (Syrphios). In one embodiment, “beneficial insects” also includes those beneficial insects preying on other insects or organisms (e.g. aphids, mites, larvae, eggs, etc) which feed off transgenic plants of the present invention.

As used herein, the term “bee” is defined as any of several winged, hairy-bodied, usually stinging insects of the superfamily Apoidea in the order Hymenoptera, including both solitary and social species and characterized by sucking and chewing mouthparts for gathering nectar and pollen. Exemplary bee species include, but are not limited to Apis, Bombus, Trigona, Osmia and the like. In one embodiment, bees include, but are not limited to bumblebees (Bombus terrestris) and honeybees (Apis mellifera).

As used herein, the term “colony” is defined as a population of dozens to typically several tens of thousand honeybees that cooperate in nest building, food collection, and brood rearing. A colony normally has a single queen, the remainder of the bees being either “workers” (females) or “drones” (males). The social structure of the colony is maintained by the queen and workers and depends on an effective system of communication. Division of labor within the worker caste primarily depends on the age of the bee but varies with the needs of the colony. Reproduction and colony strength depend on the queen, the quantity of food stores, and the size of the worker force. Honeybees can also be subdivided into the categories of “hive bees”, usually for the first part of a workers lifetime, during which the “hive bee” performs tasks within the hive, and “forager bee”, during the latter part of the bee's lifetime, during which the “forager” locates and collects pollen and nectar from outside the hive, and brings the nectar or pollen into the hive for consumption and storage.

As used herein, the term “insect pathogen” is defined as a nucleic acid-containing agent capable of proliferation within the insect or insect colony (for example, in the case of social insects), the pathogen causing disease in the insect or insect colonies, especially, but not exclusively, a virus, a bacteria, a fungus, or a mite. An insect or insect colony pathogenic agent can be an intracellular or extra-cellular parasite, and can even be a mite such as the parasitic Varroa mite, or another insect, which preys on the insect or insect colony. According to one embodiment of the invention, the pathogen is a “bee pathogen”, causing or facilitating a bee or bee colony disease, such as Colony Collapse Disorder, Sacbrood virus disease, Deformed Wing Disease, Cloudy Wing Disease, Chronic Paralysis, Nosemosis, American Foul Brood, bee and/or bee colony, Varroa infestation and the like.

As used herein, the terms “insect disease” or “insect colony disease” are defined as undesirable changes in the behavior, physiology, morphology, reproductive fitness, economic value, pollination capability, resistance to infection and/or infestation of an insect, a population of insects and/or an insect colony, directly or indirectly resulting from contact with an insect or insect colony pathogenic agent. According to one embodiment of the invention, the insect is a bee and the undesirable changes include, but are not limited to behavior, physiology, morphology, reproductive fitness, economic value, honey production, pollination capability, resistance to infection and/or infestation of the bee or bee colony.

As used herein, the term “tolerance” is defined as the ability of an insect or insect colony to resist and/or endure infestation by and/or proliferation of an insect pathogen, including, but not limited to, degree of infection, severity of symptoms, infectivity to other individuals (contagion), and the like. Tolerance can be assessed, for example, by monitoring insect longetivity/life-span, infectivity, presence of symptoms, such as, but not limited to, hunger, vitality, flight range, etc, presence of pathogenic organisms, or time course of a disease in a population following a challenge with the insect pathogen.

As used herein, the term “susceptibility” is defined as the ability of an insect or insect colony to become infested or infected by and/or support proliferation of an insect pathogen, including, but not limited to, degree of infection, severity of symptoms, infectivity to other individuals (contagion), and the like. Susceptibility can be assessed, for example, by monitoring infectivity, presence of symptoms, such as, but not limited to, hunger, vitality, flight range, etc, presence of pathogenic organisms, mortality or time course of a disease in an individual bee or bee population following a challenge with the insect pathogen.

As used herein, the term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in plant or in a plant organ, tissue or cell culture. The term “plant organ” or “plant tissue” refers to plant tissue or a group of tissues that constitute a morphologically and functionally distinct part of a plant. The term “genome” refers to the following: (1) the entire complement of genetic material (genes and non-coding sequences) that is present in each cell of an organism, or virus or organelle; and/or (2) a complete set of chromosomes inherited as a (haploid) unit from one parent. “Progeny” comprises any subsequent generation of a plant. Plant can be monocots and dicots, cereal grains, vegetables, ornamentals, crop plants and conifers. A non-limiting list of plants suitable for use with some embodiments of the present invention is provided hereinbelow.

As used herein, the phrase “vicinity” or “vicinity of said bee” refers to a location in sufficient proximity of the insect in order that the insect, or bee, when foraging or seeking to feed, could easily contact and ingest plant tissues, organs, secretions or cells. One of skill in the art would easily be able to determine how far from the origin of the bees the transgenic plants of the present invention should be located, in order to achieve efficient contact between the insects and the transgenic plants. It will be appreciated that “in the vicinity” for flying beneficial insects, such as bees, will be at a greater distance from the source of bees than, for example, “in the vicinity” for crawling insects. Further, in some embodiments, transgenic plants expressing pathogen specific dsRNA of the present invention will be planted among other crops, while in other embodiments, the transgenic plants are crop plants in themselves. It will be appreciated that according to some embodiments of the present invention, at least a portion of the plant or plant tissue or secretion (for example, nectar, pollen, leaves, exudate, and the like) is provided to the insect such as by way of ingestion, inhalation, etc.

A non-limiting list of exemplary disease-causing pathogens, and diseases of insects and insect colonies associated with the insect pathogenic agents, suitable for treatment according to some embodiments of the methods and compositions of the present invention is found in Table II below. The complete genomes of several known isolates of IAPV and information on possible phylogenic relationships between strains that can be similarly targeted with the methods and compositions of the present invention are provided in Palacios et al. 2008 (Journal of Virology, 2008, 82:6209-217). A draft of the genome of N. cerana has been provided in nCornma et al. (PLoS Pathogen, 2009; 5:e1000466). The complete genome of Varroa destructor was provided by Ongus et al (J Gen Virol 2004; 85: 3747-55), and it's mitochondrial genome by Navajas et al (Mol Biol Evol, 2002; 19:2313-17).

TABLE II
Exemplary Insect Pathogens

| Parasitic Organism | Genes |
|---|---|
| Acute bee | Acute bee paralysis virus, complete genome |
| paralysis virus | Accession NC_002548 (seq id no: 256736) |
| Israel acute | Accession: NC_009025, 14oland acute paralysis virus of bees, complete genome (seq id |
| paralysis virus | no: 256744) |
| Deformed wing | Deformed wing virus, complete genome. |
| virus | Accession NC_004830 (seq id no: 256738) |
| Kashmir bee | Accession: AY275710, 14oland14 bee virus, complete genome (seq id no: 256737) |
| virus | |
| Black queen cell | Black queen cell virus strain 14oland-6 non-structural polyprotein |
| virus | and structural polyprotein genes, complete cds. |
| | Accession: EF517521 (seq id no: 256748) |
| Chronic | Chronic bee paralysis virus rna 2, complete sequence. |
| paralysis virus | Accession: NC_010712 (seq id no: 256751) |
| Cloudy wing | Cloudy wing virus rna polymerase (pol) gene, partial cds. |
| virus | Accession AF034543 (seq id no: 256735) |
| Paenibacillus | Accession: NZ_AARF01000646, whole genome (shotgun) sequenced. (seq id no: |
| larvae (American | 256739) |
| Foul Brood) | |
| Melissococcus | Accession: EF666055 Melissococcus plutonius superoxide dismutase (soda) gene (seq |
| pluton (European | id no: 256749) |
| Foul Brood) | |
| Ascophaera apis | No genomic data |
| (Chalkbrood) | |
| Nosema apis, | 1) Accession DQ996230 (seq id no: 256743), Nosema apis RNA polymerase II largest |
| | subunit |
| | 2) Accesions EU545140 (seq id no: 256750), EF584425 (seq id no: 256747), EF584423 |
| | (seq id no: 256746), EF584418 (seq id no: 256745) all are 16S ribosomal RNA |
| | gene |
| Nosema cerana | EF091883 (seq id no: 256740), EF091884 (seq id no: 256741), and EF091885 (seq id |
| | no: 256742) are accessions of 5S ribosomal RNA gene, intergenic spacer, and small |
| | subunit ribosomal RNA gene; See Table III |
| Varroa | Accession: NC_004454 (SEQ ID NO: 311551 and 11) NADH dehydrogenase, subunit |
| destructor | 2; Accession: NC_004454 (SEQ ID NO: 12) ATP synthetase; subunit 8; Accession: |
| | NC_004454 (SEQ ID NO: 14); ATP synthetase; subunit 6; Accession: Genbank |
| | accession No. AY259834 (SEQ ID NO: 13 and 311550) Sodium channel gene; |
| | Genbank accession No. NC_004454 (SEQ ID NO: 311549) Cytochrome oxydase |
| | subunit I. |

As used herein, the phrase “Varroa destructor mite” refers to the external parasitic mite that attacks honey bees Apis cerana and Apis mellifera. The mite may be at an adult stage, feeding off the bee, or at a larval stage, inside the honey bee brood cell.

As used herein, the term “Nosema” is defined as any organism of a genus (the type of the family Nosematidae) of microsporidian protozoans that includes various parasites, particularly those causing disease in bees or bee colonies. Nosema infecting bee include but are not limited to N. cerana and N. apis. Infection of bees or bee colonies with a Nosema parasite is commonly known as Nosemosis. It will be appreciated that the gene silencing mechanisms described herein can be effective for combating microsporidean species infecting insect hosts other than bees, with examples including but not limited to, the silk moth. However, due to the heterogeneity of RNAi and metabolic pathways in microsporidia, and in view of the critical role of the individual parasite's endogenous RNAi pathways for effective gene silencing, identification of effective candidate target genes may be required in each case.

As used herein, the term “mitosome” is defined as a double membrane-bound mitochondria-like organelle of Microsporidia highly reduced from the perspective of both physical size and biochemical complexity. Mitosomal proteins include, but are not limited to protein and metabolite import proteins (TOM70, TIM22, TOM40, Imp2, mitochondrial Hsp70, and ATM1-ABC transporter proteins), proteins involved in ISC assembly and export (frataxin, ferredoxin, ISCU, ISCS, ERV1, and ferredoxin NADPH oxido-reductase [FNR]), pyruvate dehydrogenase subunits, PDHα and -β, mitochondrial glycerol-3-phosphate dehydrogenase (mtG3PDH) and manganese-containing superoxide dismutase (MnSOD).

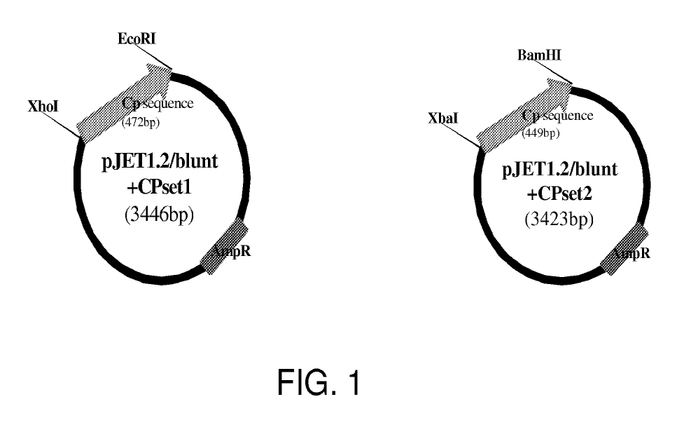

As used herein, the term “ATP/ADP transporter protein” refers to a protein which function is transfer of ATP and/or ADP through membranes. Microsporidian ATP/ADP transporter proteins homologous to ATP/ADP transporter proteins from other species have been identified in E. cuniculi and N. cerana (see above, and FIG. 1). As used herein, “putative” ATP/ADP transporter proteins, “ATP/ADP transporter protein orthologues” or “ATP/ADP transporter protein homologues” refer to proteins, or putative encoded polypeptides from polynucleotide sequences, having significant amino acid identity or similarity to known ATP/ADP transporter proteins of other species, and/or possessing consensus sequences common to known ATP/ADP transporter proteins. Examples of Nosema ATP/ADP transporter proteins, or ATP/ADP transporter protein homologues, are nc123 (SEQ ID NO:19), nc006 (SEQ ID NO:22), nc014 (SEQ ID NO:21) and nc017 (SEQ ID NO:20).

As used herein, the term “downregulating expression” is defined as causing, directly or indirectly, reduction in the transcription of a desired gene, reduction in the amount, stability or translatability of transcription products (e.g. RNA) of said gene, reduction in translation of the polypeptide(s) encoded by the desired gene and/or reduction in the amount, stability, or alteration of biochemical function of the polypeptides encoded by the desired gene, so as to reduce the amount or function of the gene products. As used herein, “downregulating expression” also relates to reduction in amount, stability or translatability of insect pathogen RNA molecules in cells of an insect, where the insect pathogen genome is a single stranded RNA molecule, as in case of a single-stranded RNA virus. Downregulating expression of a gene or other insect pathogen RNA can be monitored, for example, by direct detection of gene transcripts (for example, by PCR), by detection of polypeptide(s) encoded by the gene or insect pathogen RNA (for example, by Western blot or immunoprecipitation), by detection of biological activity of polypeptides encode by the gene (for example, catalytic activity, ligand binding, and the like), or by monitoring changes in a cell or organism resulting from reduction in expression of a desired gene or insect pathogen RNA (for example, reduced proliferation of a pathogen, reduced virulence of a pathogen, reduced motility of a cell, reduced response of a cell or organism to stimulus, etc). As used herein, the downregulation can be transient, for example, for the duration of the presence of a downregulating agent, or permanent, resulting in reduction of gene expression or insect pathogen RNA for the lifetime of the organism and/or its future generations.

Downregulation of insect pathogen polypeptides can be affected on the genomic and/or the transcript level using a variety of molecules which interfere with transcription and/or translation (e.g., RNA silencing agents, Ribozyme, DNAzyme and antisense). Treatment and prevention of plant viral infections with dsRNA has been disclosed by WO/2003/004649 to Tenllado et al. Use of dsRNA in insects is disclosed in US Patent Application 2007 0250947, US Patent Application 2006 0272049, PCT Applications WO 2007/080127 and WO 2007/080126, US patent application 20030150017, US Patent Application No. 2010068172, PCT patent application WO 02/14472, US Patent Application 20030154508, PCT application WO2009/060429, PCT patent application WO 2004/005485, PCT application WO 99/32619 and U.S. Pat. No. 6,326,193.

As used herein, the phrase “gene product” refers to proteins, peptides and RNA molecules (i.e. polynucleotides, for example, functional RNA molecules tRNA, rRNA, mRNA). Generally, the gene product which expression thereof is downregulated is a gene product of a gene critical to the function and vitality of the insect pathogen. Examples of such gene products include nucleic acids (such as viral RNA), proteins, peptides, glycoproteins and lipoproteins normally produced by an insect pathogen. For example, gene products which may be downregulated in the insect pathogens include transcripts of genes associated with pathogen replication, pathogen growth, pathogen energy metabolism, pathogen resistance to host defense mechanisms, pathogen motility, and the like. Specific, non-limiting example are the ATP/ADP transporter proteins and mitosomal proteins of Nosema.

Following is a list of agents capable of downregulating expression level and/or activity of insect pathogen gene products, suitable for use with the methods of the present invention.

Downregulation of expression of insect pathogen gene products can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding insect pathogen gene. RNA silencing has been observed in many types of organisms, including plants, animals, and fungi.

As used herein, the term “nucleic acid agent downregulating expression of a gene product” or “RNA silencing agent” refers to an RNA which is capable of inhibiting or “silencing” the expression of a target gene. In certain embodiments, the nucleic acid agent is capable of preventing complete processing (e.g., the full translation and/or expression) of an mRNA molecule through a post-transcriptional silencing mechanism. Nucleic acid agent includes noncoding RNA molecules, for example RNA duplexes comprising paired strands, as well as precursor RNAs from which such small non-coding RNAs can be generated. Exemplary nucleic acid agents include dsRNAs such as siRNAs, miRNAs and shRNAs. In one embodiment, the nucleic acid agent is capable of inducing RNA interference. In another embodiment, the nucleic acid agent is capable of mediating translational repression.

RNA interference commonly refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as DICER. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to down-regulate protein expression from mRNA.

According to one embodiment, the dsRNA is greater than 30 bp. The use of long dsRNAs can provide numerous advantages in that the cell can select the optimal silencing sequence alleviating the need to test numerous siRNAs; long dsRNAs will allow for silencing libraries to have less complexity than would be necessary for siRNAs.

In one embodiment of the present invention, the dsRNA is greater than 30 base-pairs, and is selected from the group complementary to a sequence as set forth in SEQ ID NOs: 55-59. In another embodiment of the present invention, the dsRNA is greater than 30 base pairs and comprises at least two sequences complementary to a sequence selected from group as set forth in SEQ ID NOs: 256752, 256761, 256762 and 311552. In yet another embodiment, the dsRNA comprises the sequence complementary to the sequence as set forth in SEQ ID NO: 256752 and to a sequence as set forth in at least one of SEQ ID NOs: 256761, 256762 and 311552. In one embodiment of the present invention, the dsRNA comprises at least two sequences downregulating expression of a target insect pathogen gene, wherein the at least two sequences can be contiguous, or non-contiguous with respect to one another.

Another method of downregulating expression of insect pathogen gene products is by introduction of small inhibitory RNAs (siRNAs).

The term “siRNA” refers to small inhibitory RNA duplexes (generally between 18-30 basepairs, between 19 and 25 basepairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA) that is capable of folding back onto itself to form a double stranded structure. Thus, as mentioned the nucleic acid agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. In specific embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 15, 10 nucleotides or less. In some embodiments, the number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop are detailed in Brummelkamp, T. R. et al. (2002) Science 296: 550 and Castanotto, D. et al. (2002) RNA 8:1454. It will be recognized by one of skill in the art that the resulting single chain oligonucleotide forms a stem-loop or hairpin structure comprising a double-stranded region capable of interacting with the RNAi machinery.

The first and second regions of the hairpin RNA molecule comprise the base-paired stem of the hairpin structure. The first and the second regions are inverted repeats of one another and share sufficient complementarity to allow the formation of the base-paired stem region. In specific embodiments, the first and the second regions are fully complementary to one another. Alternatively, the first and the second regions may be partially complementary to each other so long as they are capable of hybridizing to one another to form a base-paired stem region. The amount of complementarity between the first and the second regions can be calculated as a percentage of the entire region. Thus, the first and the second regions of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the second regions are at least about 1000, 500, 400, 300, 200, 100, 50, 40, 30, 25, 22, 20, 15 or 10 nucleotides in length. In specific embodiments, the length of the first and/or the second regions is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides. In other embodiments, the length of the first and/or the second region comprises at least 10-20 nucleotides, 20-35 nucleotides, 30-45 nucleotides, 40-50 nucleotides, 50-100 nucleotides, or 100-300 nucleotides. See, for example, International Publication No. WO 0200904. In specific embodiments, the first and the second region comprise at least 20 nucleotides having at least 85% complementary to the first segment. In still other embodiments, the first and the second region which form the stem-loop structure of the hairpin comprises 3′ or 5′ overhang regions having unpaired nucleotide residues.

In specific embodiments, the sequences used in the first, the second, and/or the second regions comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the nucleic acid. Thus, in some embodiments of the invention, the first, second and/or second region of the nucleic acid comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 20-35 nucleotides, about 25-50 nucleotides, about 20 to 75 nucleotides, about 40-90 nucleotides about 15-100 nucleotides.

In specific embodiments, the domain of the first, the second, and/or the second region has 100% sequence identity to the target polynucleotide. In other embodiments, the domain of the first, the second and/or the second region having homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the second regions to the target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

The amount of complementarity shared between the first, second, and/or second region and the target polynucleotide or the amount of complementarity shared between the first region and the second region (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression.

Any region of the target polynucleotide can be used to design the domain of the nucleic acid agent that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments a domain of the nucleic acid agent shares sufficient homology to at least about 15 consecutive nucleotides from about nucleotides 1-50, 50-100, 100-150, 150-200, 200-250, 250-300, 300-350, 350-400, 400-450, 450-500, 550-600, 600-650, 650-700, 750-800, 850-900, 950-1000, 1000-1050, 1050-1100, 1100-1200, 1200-1300, 1300-1400, 1400-1500, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem. 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904, herein incorporated by reference.

According to another embodiment the nucleic acid agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

According to one embodiment of the present invention, the nucleic acid agent is capable of causing cleavage and/or degradation of target insect pathogen transcribed polynucleotide sequences. As used herein, the phrases “target” or “target polynucleotide sequence” refer to any sequence present in an insect pathogen cell, or infected host cell, whether naturally occurring sequence or a heterologous sequence present, which insect pathogen transcribed polynucleotide sequence(s) has(ve) a function that is desired to be reduced or inhibited. The insect pathogen target sequence may be a coding sequence, that is, it is translated to express a protein or a functional fragment thereof. Alternatively, the target sequence may be non-coding, but may have a regulatory function, or it may be without any known function. The term “gene” is intended to include any target sequence intended to be “silenced”, whether or not transcribed and/or translated, including regulatory sequences, such as promoters, enhancers and other non-coding sequences.

In one embodiment of the present invention, synthesis of nucleic acid agents suitable for use with the present invention can be effected as follows. First, the insect pathogen polypeptide mRNA or other target sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl Chem Biochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (see Ambion technical library 91/912 at the Ambion website).

Second, potential target sites are compared to an appropriate genomic database (e.g., human, mouse, rat, insect, etc.) using any sequence alignment software, such as the BLAST software available from the NCBI server of the NIH. Putative target sites which exhibit significant homology to other coding sequences are filtered out. For example, host (e.g. bee) and plant host target sites can be filtered out in this manner.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene or sequence for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to insect or plant host gene sequences, or non-relevant insect pathogen target sequences.

In specific embodiments, suitable insect pathogen specific siRNA can be an IAPV, Nosema or Varroa specific siRNA, corresponding to IAPV sequences SEQ ID NOs: 256752, 256761, 256762 and 311552, Nosema cerana sequences SEQ ID NOs: 55-59, and Varroa destructor sequences SEQ ID NOS: 253194, 254090 and 259289. Additional suitable bee pathogen siRNAs can be designed according to sequences from any bee pathogens, for example, the sequences detailed in Table II, including, but not limited to Acute Bee Paralysis Virus (for example, SEQ ID NOs: 289158-298614), Deformed Wing Virus (for example, SEQ ID NOs: 9533-276380), Kashmir Bee Virus (for example, SEQ ID NOs: 299009-308499), Black Queen Cell Virus (for example, SEQ ID NOs: 276381-284662), Chronic Paralysis Virus (for example, SEQ ID NOs: 284663-286947), Cloudy Wing Virus (for example, SEQ ID NOs: 286948-287341), Paenibacillus larvae (for example, SEQ ID NOs: 287342-288735), Melissococcus pluton (for example, SEQ ID NOs: 288736-289157), Nosema apis (for example, SEQ ID NOs: 308500-311548).

Additional suitable Nosema siRNAs can be designed according to sequences from any Nosema sequence, for example, the sequences detailed herein, including, but not limited to TOM70 (for example, SEQ ID NOs:60-11687), TIM22, TOM40 (for example, SEQ ID NOs: 11688-19103), Imp2, mitochondrial Hsp70 (for example, SEQ ID NOs: 19104-34511), ATM1-ABC transporter proteins (for example, SEQ ID NOs: 34512-80411), Frataxin, Ferredoxin, ERV1 (for example, SEQ ID NOs: 80412-84803), ferredoxin, NADPH oxido-reductase [FNR] (for example, SEQ ID NOs:84804-94911, 94912-108140), pyruvate dehydrogenase α subunit (for example, SEQ ID NOs: 108141-116852), pyruvate dehydrogenase β subunit (for example, SEQ ID NOs: 116853-125294), mitochondrial glycerol-3-phosphate dehydrogenase (mtG3PDH) (for example, SEQ ID NOs: 125295-140999), manganese-containing superoxide dismutase (MnSOD) (for example, SEQ ID NOs: 141000-146687), DNAJ (Hsp70 interacting) (for example, SEQ ID NOs: 146688-157505), Iron Sulfur cluster ISU1, Cystein desulfurase Nsf1 (for example, SEQ ID NOs: 157506-169079), NAR1 (for example, SEQ ID NOs:169080-178790), RLI1 (for example, SEQ ID NOs:178791-195062), NC006 (for example, SEQ ID NOs: 195063-209633), NC123 (for example, SEQ ID NOs: 209634-224609), NC014 (for example, SEQ ID NOs: 224610-239747) and NC017 (for example, SEQ ID NOs:239748-252698) (see Table III, below).

TABLE III

| | Encephalitozoon | Nosema | | |
|---|---|---|---|---|
| | GenBank | GenBank | Nosema GenBank (DNA)/ | |
| | (protein) | (protein) | coordinates | SEQ ID NO: |
| TOM70 | | EEQ82075.1 | ACOL01000120/8001-9314 | SEQ ID NO: 27 |
| TIM22 | CAD25556.1 | — | | |
| TOM40 | CAD25408.1 | EEQ82411.1 | ACOL01000061/14217-15062 | SEQ ID NO: 28 |
| Imp2 | | | | |
| mitochondrial | NP_586360 | EEQ81757 | ACOL01000228.1/4051-5784 | SEQ ID NO: 29 |
| Hsp70 | | | | |
| ATM1-ABC | CAD26030 | EEQ82581.1 | ACOL01000042/1310-3019 | SEQ ID NO: 30 |
| transporter | | EEQ82586.1 | ACOL01000042/8786-10507 | SEQ ID NO: 31 |
| proteins | | EEQ82587.1 | ACOL01000042/10905-12638 | SEQ ID NO: 32 |
| Frataxin | XP_965969 | — | | |
| Ferredoxin | NP_585988.1\| | — | | |
| ERV1 | CAD25469 | EEQ82883 | ACOL01000016/5063-5572 | SEQ ID NO: 33 |
| ferredoxin | CAD27143 | EEQ81930 | ACOL01000159/6191-7324 | SEQ ID NO: 34 |
| NADPH | | | | |
| oxido- | | EEQ83026 | ACOL01000006/2688-4190 | SEQ ID NO: 35 |
| reductase | | | | |
| [FNR] | | | | |
| pyruvate | CAD27078 | EEQ82465 | ACOL01000055/12839-13828 | SEQ ID NO: 36 |
| dehydrogenase | CAD25304 | EEQ81634 | ACOL01000316/888-1847 | SEQ ID NO: 37 |
| subunits, | | | | |
| PDHα and -β | | | | |
| mitochondrial | CAD25806 | EEQ82606 | ACOL01000039/538-2304 | SEQ ID NO: 38 |
| glycerol-3- | | | | |
| phosphate | | | | |
| dehydrogenase | | | | |
| (mtG3PDH) | | | | |
| manganese- | CAD26018 | EEQ82623 | ACOL01000038/17225-17878 | SEQ ID NO: 39 |
| containing | | | | |
| superoxide | | | | |
| dismutase | | | | |
| (MnSOD) | | | | |
| DNAJ (Hsp70 | Q8SRK0 | EEQ82425 | ACOL01000059/6412-7635 | SEQ ID NO: 40 |
| interacting) | | | | |
| Iron Sulfur | Q8SSM2 | — | | |
| cluster ISU1 | | | | |
| Cystein | Q8SQS2 | EEQ82825 | ACOL01000020/5924-7231 | SEQ ID NO: 41 |
| desulfurase | | | | |
| Nsf1 | | | | |
| Critical Fe/S | | | | |
| cytosolic | | | | |
| proteins | | | | |
| NAR1 | NP_597440.1 | EEQ82578 | ACOL01000043/12613-13713 | SEQ ID NO: 42 |
| RLI1 | | EEQ83099 | ACOL01000003/27828-29657 | SEQ ID NO: 43 |
| ATP/ADP | | | | |
| Transporter | | | | |
| homologues | | | | |
| Nc006 | | EEQ83030.1 | ACOL01000006/9068-10708 | SEQ ID NO: 44 |
| Nc123 | | EEQ82057.1 | ACOL01000123/4795-6480 | SEQ ID NO: 45 |
| Nc014 | | EEQ82913.1 | ACOL01000014/4714-6417 | SEQ ID NO: 46 |
| Nc017 | | EEQ82872.1 | ACOL01000017/9811-11271 | SEQ ID NO: 47 |

Multiple insect-pathogen sequences can be designed to include sequences suitable for producing siRNAs effective against more than one insect pathogen. Such multiple insect-pathogen dsRNA can be of the long or short variety, and may include sequences corresponding to homologous sequences within a class of insect pathogens (multiple bee-virus sequences, for example), or sequences corresponding to diverse classes of pathogens (e.g. viral+bacterial+fungal+mite sequences, etc). Further, multiple sequences can be designed to include two or more dsRNA sequences of the same bee-pathogen.

Primer sequences for producing nucleic acid agents for silencing expression of some of the pathogen genes are detailed in Table IV.

TABLE IV
Insect pathogen-specific dsRNA sequences for silencing pathogen
genes and the primer sequences used to make them:

| IAPV | SEQ ID NO: | GenBank locator | Size |
|---|---|---|---|
| IAPV: dsRNA synthesis | | | |
| F: TAATACGACTCACTATAGGGCGACCA | 256767 | 8977-9835 | 408 |
| CCCCTCTCAAACAATCTCAAACA | | (NC_009025) | (excluding |
| R: TAATACGACTCACTATAGGGCGATAT | 256768 | | the T7 |
| ATCCAGTTCAAGTGTCGGTTTTC | | | promoter |
| | | | (in bold)) |
| IAPV: dsRNA synthesis | | | |
| F: TAATACGACTCACTATAGGGCGAGAC | 256769 | 6168-6594 | 427 |
| ACAATTCTTGAAATGCCAAACT | | (NC_009025) | (excluding |
| R: TAATACGACTCACTATAGGGCGACAT | 256770 | | the T7 |
| GTGTTACCATACGACTGCTGTAA | | | promoter |
| | | | (in bold)) |

NOSEMA

| Primer name | Sequence (SEQ ID NO) |
|---|---|
| NA7001 T7 Nc0006F | CTAATACGACTCACTATAGGGAGACAGCTAACGAGCCCGTTTC |
| | (SEQ ID NO: 1) |
| NA7002 T7 Nc006 R | CTAATACGACTCACTATAGGGAGACCATAGTAATCCATCCACTAC |
| | (SEQ ID NO: 2) |
| NA7003 T7 Nc123 F | CTAATACGACTCACTATAGGGAGACTGGTCTTTAACGAATGGAC |
| | (SEQ ID NO: 3) |
| NA7004 T7 Nc123 R | CTAATACGACTCACTATAGGGAGAGTGGGCACGCTATGGCAAC |
| | (SEQ ID NO: 4) |
| NA7005 T7 Nc014 F | CTAATACGACTCACTATAGGGAGACTCCTGGACAGTCCGCTAG |
| | (SEQ ID NO: 5) |
| NA7006 T7 Nc014 R | CTAATACGACTCACTATAGGGAGAATCAGTTGACGGTAAACGG |
| | (SEQ ID NO: 6) |
| NA7007 T7 Nc017 F | CTAATACGACTCACTATAGGGAGAGCTTGATGGGCTTATCTCC |
| | (SEQ ID NO: 7) |
| NA7008 T7 Nc017 R | CTAATACGACTCACTATAGGGAGAGCAATGCGATTTCCACGG |
| | (SEQ ID NO: 8) |
| TOM-70 PROTEIN | |
| NT7009 T7 TOM70 F | CTAATACGACTCACTATAGGGAGACTGAATGTTACAAGCAGATGGG |
| | (SEQ ID NO: 9) |
| NT7015 TOM70 R | CTAATACGACTCACTATAGGGAGAACCAGGAGTATCTGGATGAC |
| | (SEQ ID NO: 10) |

*Note:
The Nosema specific sequences are in plain font.

Additional suitable Varroa siRNAs can be designed according to sequences from any Varroa sequence, for example, the sequences detailed herein, including, but not limited to SEQ ID NO: 252699-256728.

It will be appreciated that the dsRNA sequences target RNA transcripts complementary to pathogen DNA or RNA (in the case of a virus) sequences of the targeted gene which are expressed in the parasite (transcribed into RNA), and that the actual complementation taking place in the RNAi pathways occurs following reduction of the dsRNA to smaller fragments by the RNAi enzymes.

Multiple insect pathogen-specific siRNA sequences can be designed to include sequences suitable for producing siRNAs effective against more than one pathogenic species. Such multiple insect pathogen-specific dsRNA can be of the long or short variety, and may also be combined with sequences corresponding to diverse classes of pathogens (e.g. viral and/or bacterial and/or fungal sequences, etc). Further, multiple sequences can be designed to include two or more dsRNA sequences of the same pathogen. Thus, in some embodiments, the nucleic acid construct comprises at least two nucleic acid agents, each of said nucleic acid agents downregulating expression of at least one gene product of at least one insect pathogen. In some further embodiments the at least two nucleic acid agents downregulate expression of the same target gene product. In yet further embodiments the at least two nucleic acid agents down-regulate expression of at least two different target gene product.

It will be appreciated that the RNA silencing agent of the present invention need not be limited to those molecules containing only RNA, but further encompasses chemically-modified nucleotides and non-nucleotides.

Another agent capable of downregulating an insect pathogen gene product is a DNAzyme molecule capable of specifically cleaving an mRNA transcript or DNA sequence of the insect pathogen gene. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al.

Downregulation of insect pathogen polypeptides or cleavage of insect pathogen RNA can also be effected by using an antisense polynucleotide capable of specifically hybridizing with an mRNA transcript encoding the insect pathogen polypeptide or an insect pathogen RNA target sequence.

Design of antisense molecules which can be used to efficiently downregulate an insect pathogen polypeptide must be effected while considering two aspects important to the antisense approach. The first aspect is delivery of the oligonucleotide into the cytoplasm of the appropriate cells, while the second aspect is design of an oligonucleotide which specifically binds the designated mRNA or RNA target sequence within cells in a way which inhibits translation thereof.

A number of delivery strategies which can be used to efficiently deliver oligonucleotides into a wide variety of cell types have been disclosed [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)]. Several approaches for designing and predicting efficiency of specific oligonucleotides using an in vitro system were also published (Matveeva et al., Nature Biotechnology 16: 1374-1375 (1998)].

For example, a suitable antisense oligonucleotide targeted against an insect pathogen would be of the sequences complementary to pathogen mRNA sequences as set forth in SEQ ID NOs: 55-59, 253194, 254090, 259289, 256752, 256761.

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating an insect pathogen polypeptide is a ribozyme molecule capable of specifically cleaving an mRNA transcript encoding an insect pathogen polypeptide. Ribozymes are being increasingly used for the sequence-specific inhibition of gene expression by the cleavage of mRNAs encoding proteins of interest [Welch et al., Curr Opin Biotechnol. 9:486-96 (1998)]. Ribozymes have been identified in insects (Webb et al, Science 2009; 326:953), and used effectively for gene silencing in insects (Lee et al, FASEB J 2001; 15:2390-400).

An additional method of regulating the expression of an insect pathogen gene in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

In some embodiments of the present invention, the methods and compositions of the invention employ nucleic acid elements that when transcribed “form” a dsRNA molecule. Accordingly, the heterologous polynucleotide being expressed need not form the dsRNA by itself, but can interact with other sequences in the plant cell or in the insect gut, or even in the pathogen after ingestion to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In such an embodiment, the dsRNA is “formed” when the target for the miRNA or siRNA interacts with the miRNA present in the cell. The resulting dsRNA can then reduce the level of expression of the gene or genes to be silenced. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

It will be appreciated that mechanisms other than dsRNA for targeting the pathogens proteins or other gene products can effectively block gene expression in the parasite, and therefore potentially reduce insect pathogen, severity of symptoms and contagiousness in the host insect. Any molecules capable of traversing the membrane of insect mucosal epithelial cells and able to disrupt pathogen gene expression or activity, could potentially enter the pathogen in an early stage, and reduce infection levels and severity of symptoms in infected hosts.

The nucleic acid agent of the present invention is expressed in plant cells, plants, plant tissues and/or organs. For transcription from a transgene in vivo or from an expression cassette, a regulatory region (e.g., promoter, enhancer, silencer, leader, intron and polyadenylation) may be used to modulate the transcription of the RNA strand (or strands). Therefore, in one embodiment, there is provided a nucleic acid construct comprising a polynucleotide encoding a nucleic acid agent downregulating expression of a gene product of an insect pathogen, the polynucleotide operably linked to a cis-regulatory element operable in plants.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide encoding the nucleic acid agent employed in the methods and compositions of the invention can be provided in expression cassettes for expression in a plant or organism of interest. It is recognized that multiple nucleic acid agents including multiple identical nucleic acid agents, multiple nucleic acid agents targeting different regions of the target sequence, or multiple nucleic acid agents from different target sequences can be used. In this embodiment, it is recognized that each nucleic acid agent can be contained in a single or separate cassette, DNA construct, or vector. As discussed, any means of providing the nucleic acid agent is contemplated. A plant or plant cell can be transformed with a single cassette comprising DNA encoding one or more nucleic acid agent or separate cassettes comprising each nucleic acid agent can be used to transform a plant or plant cell or host cell. Likewise, a plant transformed with one component can be subsequently transformed with the second component. One or more nucleic acid agent can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

“Regulatory sequences” or “control elements” refer to nucleotide sequences located upstream, within, or downstream of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, termination sequences, pausing sequences, polyadenylation recognition sequences, and the like.

The expression cassette will include in the 5′-3′ direction of transcription, a transcriptional and translational initiation region (i.e., a promoter), a polynucleotide comprising the silencing element employed in the methods and compositions of the invention, and a transcriptional and translational termination region (i.e., termination region) functional in plants. The regulatory regions (i.e., promoters, transcriptional regulatory regions, and translational termination regions) and/or the polynucleotides employed in the invention may be native/analogous to the host cell or to each other. Alternatively, the regulatory regions and/or the polynucleotide employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the nucleic acid agent, may be native with the plant host, or may be derived from another source (i.e., foreign or heterologous) to the promoter, the polynucleotide comprising silencing element, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The polynucleotide encoding the nucleic acid agent can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of contact with the beneficial insect. Of particular interest are nectary specific, pollen specific, and flower-specific promoters (see Fernandez et al, Plant Phys 2009, 151:1729-40 and Kram et al, BMC Plant Biol 2009; 9:92) such as, but not limited to the tapetal-oleosin like protein promoter (see U.S. Pat. No. 7,303,917 to Robert), the chsA (chalcone synthase) promoter from Petunia hybrida, promoters from scent biosynthetic enzymes, such as the Linalool Synthase (LIS) promoter from Clarkia brewri, the 1,8-cineole synthase and (+)-sabinene synthase promoters from common sage (U.S. Pat. No. 5,891,697), the Benzyl alcohol:acetyl CoA acetyltransferase (BEAT) promoter, the S-adenosyl-L-methionine:(iso)eugenol O-methyltransferase, (IEMT) promoter, the Limonene synthase's nucleotide promoter (U.S. Pat. No. 5,871,988), the S-Adenosyl-L-Methionine:Salicylic Acid Methyl Transferase (S-AMT) and the S-Adenosyl-L-methionine:benzoic acid carboxyl methyltransferase (BAMT) promoter, the NEC1 nectary specific promoter from Petunia hybrida, fruit and flower specific promoters PPC2, TPRP, IMA, CRC and PG (Fernandez et al, 2009), the Aribidopsis nectarium gene cluster promoters (Kram et al, BMC Plant Biol, 2009), the promoters from nectary-related LEAFY, UFO, AGAMOUS, SHATTERPROOF1/2, APETALA2/3, PISTILLATA, and SEPALLATA1/2/3 genes (Kram et al, 2009), the tobacco NECTARIN I, III and V promoters, the Brassica NTR1 promoter and the Arabidopsis sesquiterpene synthetase promoter.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J=12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean).

In one embodiment of this invention the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells. Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the SCSV3, SCSV4, SCSV5, and SCSV7 promoters; the rolC gene promoter of Agrobacterium rhizogenes; the rolA gene promoter of Agrobacterium rhizogenes; the promoter of the Agrobacterium tumefaciens T-DNA gene 5; the rice sucrose synthase RSs1 gene promoter; the CoYMV or Commelina yellow mottle badnavirus promoter; the CFDV or coconut foliar decay virus promoter; the RTBV or rice tungro bacilliform virus promoter; the pea glutamin synthase GS3A gene; the inv CD111 and inv CD141 promoters of the potato invertase genes, the promoter of the sulfate transporter geneSultr1; a promoter of a sucrose synthase gene; and the promoter of a tobacco sucrose transporter gene and the like.

Additional possible promoters include the Black Cherry promoter for Prunasin Hydrolase (PH DL1.4 PRO) (U.S. Pat. No. 6,797,859), Thioredoxin H promoter from cucumber and rice, Rice (RSs1) and maize sucrose synthese-1 promoters, PP2 promoter from pumpkin, At SUC2 promoter, At SAM-1 (S-adenosylmethionine synthetase), and the Rice tungro bacilliform virus (RTBV) promoter.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional, non-limiting examples of selectable markers include phenotypic markers such as beta.-galactosidase and fluorescent proteins such as green fluorescent protein (GFP), cyan florescent protein (CYP), and yellow florescent protein (PhiYFP.TM. from Evrogen. For additional selectable markers, see generally, Yarranton (1992) Curr. Opin. Biotech. 3:506-511; Christopherson et al. (1992) Proc. Natl. Acad. Sci. USA 89:6314-6318, and Reines et al. (1993) Proc. Natl. Acad. Sci. USA 90:1917-1921. Such disclosures are herein incorporated by reference. The above list of selectable marker genes is not meant to be limiting. Any selectable marker gene can be used in the present invention.

It is recognized that the polynucleotides comprising sequences encoding the nucleic acid agent can be used to transform organisms to provide for host organism production of these components. Such host organisms include baculoviruses, bacteria, and the like. In this manner, the combination of polynucleotides encoding the nucleic acid agent may be introduced via a suitable vector into a microbial host, and said host applied to the environment, or to plants or animals. The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

In one embodiment, the methods of the invention involve introducing a polynucleotide or nucleic acid construct into a plant. “Introducing” is intended to mean presenting to the plant the polynucleotide or polypeptide in such a manner that the sequence gains access to the interior of a cell of the plant, or to interstitial fluid or other extracellular compartment of the plant, in such a manner as to be available to a beneficial pollinating or herbivorous insect. The methods of the invention do not depend on a particular method for introducing a sequence into a plant, only that the polynucleotide gains access to the interior of at least one cell of the plant. Methods for introducing polynucleotide into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant is introduced into a plant. Transformation protocols as well as protocols for introducing polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. No. 5,886,244; and, 5,932,782; Tomes et al. (1995) in Plant Cell, Tissue, and Organ Culture. Fundamental Methods, ed. Gamborg and Phillips (Springer-Verlag, Berlin); and Lec1 transformation (WO 00/28058). Also see U.S. Pat. Nos. 5,240,855; 5,322,783; and, 5,324,646; U.S. Pat. No. 5,736,369; all of which are herein incorporated by reference.

In specific embodiments, the nucleic acid agent and insect pathogen-specific sequences of the invention can be provided to a plant using a variety of transient transformation methods. Such transient transformation methods include, but are not limited to, the introduction of the polynucleotide or variants and fragments thereof directly into the plant or the introduction of the transcript into the plant. Such methods include, for example, microinjection or particle bombardment. See, for example, Crossway et al. (1986) Mol. Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, polynucleotides can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudoisuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis noolkatensis). In specific embodiments, plants of the present invention are crop plants (for example, corn, alfalfa, sunflower, Brassica, soybean, cotton, safflower, peanut, sorghum, wheat, millet, tobacco, etc.). In some embodiments, rapeseed (Brassica), clover and alfalfa plants are optimal.

In some embodiments, the beneficial insects are pollinating insects, such as, but not limited to, bees. In other embodiments, the beneficial insects are herbivores. Thus, in some embodiments, the beneficial insects are attracted to, make contact with, or feed off the flower or flower-related tissues of the plant. Some non-limiting examples of plants particularly suited for use of the present invention with pollinating insects are flowering plants that are ubiquitous in their distribution, flower in a season relevant for pollinating insect activity, produce nectar, are easily transformed and easily cultivated. Thus, in some specific embodiments, the plant is a flowering plant selected from the group consisting of rapeseed, clover and alfalfa. In another embodiment, plants having extrafloral nectaries are selected.

The methods detailed herein can be used to produce plants useful in combating pathogen-related disease in insects. Thus, in one specific embodiment, there is provided a method of producing a plant capable of reducing the susceptibility of an insect feeding from said plant, plant tissues or plant secretions to a disease caused by an insect pathogen, the method comprising transforming cells of said plant with a nucleic acid construct comprising a polynucleotide expressing a nucleic acid agent down-regulating expression of a gene product of an insect pathogen, said polynucleotide operably linked to a cis-regulatory element operable in plants, selecting transformed plants or plant cells expressing insect pathogen-specific dsRNA, and propagating said plants or plant cells to produce a plant or plant cells capable of reducing the susceptibility of an insect feeding from said plant.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The methods of the invention comprise methods for reducing susceptibility of an insect (e.g. a bee) to a disease caused by an insect pathogen (e.g. a bee pathogen), comprising providing to the insect plants expressing the nucleic acid construct comprising a polynucleotide expressing the nucleic acid agent of the invention, wherein said nucleic acid construct, when ingested by an insect, specifically reduces the level of a target polynucleotide of the insect pathogen and thereby controls the insect pathogen. The insect can be fed the nucleic acid agent in a variety of ways. For example, in one embodiment, the polynucleotide comprising the nucleic acid agent is introduced into a plant. As the insect feeds on the plant or part thereof expressing these sequences, the nucleic acid agent is delivered to the pathogen. When the nucleic acid agent is delivered to the plant in this manner, it is recognized that the nucleic acid agent can be expressed constitutively or alternatively, it may be produced in a stage-specific manner by employing the various inducible or tissue-preferred or developmentally regulated promoters that are discussed elsewhere herein. For example, the nucleic acid agent could be expressed in aerial plant tissues such as, the leaves, stem, flower, etc. In these embodiments, pathogens of bees and other pollinating insects, such as IAPV and Nosema can be targeted. In other embodiments, the silencing element is expressed in a root.

The polynucleotide sequences of interest used to transform plants can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be introduced at a desired genomic location using a site-specific recombination system. See, for example, WO99/25821, WO99/25854, WO99/25840, WO99/25855, and WO99/25853, all of which are herein incorporated by reference.

Methods and compositions are further provided which allow for an increase in RNAi produced from the silencing element. In such embodiments, the methods and compositions employ a first polynucleotide comprising a nucleic acid agent for a target pathogen sequence operably linked to a promoter active in the plant cell; and, a second polynucleotide comprising a suppressor enhancer element comprising the target pathogen sequence or an active variant or fragment thereof operably linked to a promoter active in the plant cell. The combined expression of the silencing element with suppressor enhancer element leads to an increased amplification of the inhibitory RNA produced from the silencing element over that achievable with only the expression of the silencing element alone. In addition to the increased amplification of the specific RNAi species itself, the methods and compositions further allow for the production of a diverse population of RNAi species and for the systemic production of RNAi throughout the plant. Thus, the various methods and compositions provide improved methods for the delivery of inhibitory RNA to the target organism. See, for example, U.S. Patent Application No. 20090265818.

It will be appreciated that the RNA, dsRNA, siRNA, or miRNA of the present invention may be produced chemically or enzymatically through manual or automated reactions or in vivo in an organism. RNA may also be produced by partial or total organic synthesis. Any modified ribonucleotide can be introduced by in vitro enzymatic or organic synthesis. The RNA may be synthesized by a cellular RNA polymerase or a bacteriophage RNA polymerase (e.g., T3, T7, SP6). If synthesized chemically or by in vitro enzymatic synthesis, the RNA may be purified prior to feeding or formulated in an acceptable carrier and provided as a liquid, solid or semi-solid to the bees. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands. In some embodiments, the nucleic acid agent of the present invention is provided to the plant from an external source, and is taken up into the plant via conducting tissues (i.e. xylem and phloem). Such solutions, or powders comprising the nucleic acid agents of the present invention can be applied by irrigation or by mechanical means such as spraying or dusting. As phloem is the source of fluid for nectar production, it is conceivable that nucleic acid agent of the present invention can be introduced into nectar and other plant secretions via external application to the plant.

In some embodiments, the methods of the present invention can be used to combat Colony Collapse Disorder, wherein the beneficial insect is a bee, and the insect pathogen-associated disease is any of IAPV, nosemosis or Varroa infestation. Thus, there is provided a method of reducing the susceptibility of honeybees to Colony Collapse Disorder, the method comprising providing to the honeybees plants expressing a nucleic acid construct comprising a polynucleotide encoding a nucleic acid agent downregulating a gene product of an insect pathogen selected from the group consisting of IAPV, Nosema and Varroa. In some specific embodiments, the nucleic acid agent is complementary to SEQ ID NOs: 256752, 256761, 256762 and 311552, 55-59 or 253194, 254090 and 259289.

It is expected that during the life of a patent maturing from this application many relevant methods and compositions comprising a nucleic acid agent down-regulating expression of a gene product of an insect pathogen will be developed and the scope of the terms nucleic acid agent of the invention, and insect pathogen-specific dsRNA is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Cloning of IAPV-Specific dsRNA into a Plant Expressible Binary Vector

dsRNA complementary to the IAPV Coat Protein sequence was found to be effective in reducing viral infection and preventing bee mortality when fed to the bees. The following details preparation of a binary vector capable of producing transgenic plants expressing IAPV specific dsRNA.

Production and Cloning of IAPV Sequences:

PCR products representing the 433 bp portion of the IAPV Coat Protein gene (position 8977-9410 in the IAPV genome) (SEQ ID NO: 311552) and it's inverted repeat sequence were prepared using the following primers and pDRIVE plasmid as a template.

For the IAPV Coat Protein sequence

1. The forward primer at position 8977 in the IAPV genome, containing XhoI restriction and T7 sequence (“XhoI/T7-CP F”):

5′-GGCTCGAGTAATACGACTCACTATAGGGCGACCAC-3′
length = 35 bp, Tm = 73° C.

2. The reverse primer at position 9410 in the IAPV genome, containing EcoRI restriction sequence (“EcoRI-CP R”):

5′CCGAATTCTATATCCAGTTCAAGTGTCGGTTTTC3′
length = 34 bp, Tm = 68° C.

PCR reaction was performed using 40 cycles of denaturation at 94° C., hybridization at 59° C. and elongation at 72° C. PCR product is a 472 bp fragment (“CPset1”), flanked by XhoI and EcoRI sites.

For the inverted repeat of the IAPV Coat Protein sequence:

1. Forward primer at position 9410 in the IAPV genome, containing BamHI restriction sequence (“BamHI-CP F”):

5′GGGGATCCTATATCCAGTTCAAGTGTC3′
length = 27 bp, Tm = 63° C.

2. Reverse primer at position 8977 in the IAPV genome, containing XbaI restriction sequence (“XbaI-CP R”):

5′CCTCTAGACCACCCCTCTACAACAATCTC3′
length = 29 bp, Tm = 65° C.

PCR reaction was performed using 40 cycles of denaturation at 94° C., hybridization at 59° C. and elongation at 72° C. PCR product is a 449 bp fragment (“CPset2”), flanked by XbaI and BamI sites.

To avoid any interference with pDRIVE for next steps, a gel extraction of the 472 and 449 bp fragments was performed.

PCR products “CPset1” and “CPset2” were ligated into plasmid pJET1.2 according to manufacturer's instructions (CloneJET™, Fermentas), which were amplified in E. coli, then selected for Ampicillin resistance. Recombinant colonies with plasmids CPset1 (pJET-CPset1) and CPset2 (pJET-CPset2) were selected, grown and plasmid DNA isolated (FIG. 1).

The cloned sequence “CPset1” was released by digestion with the appropriate restriction enzymes, and ligated into appropriately double-digested pRNA69 plasmids, according to the manufacturer's protocol (Genaid Biotech Ltd, Taiwan). The pRNA69 plasmid includes the strong 35S CaMV promoter and the octopine terminator sequences for expression of cloned sequences in plants.

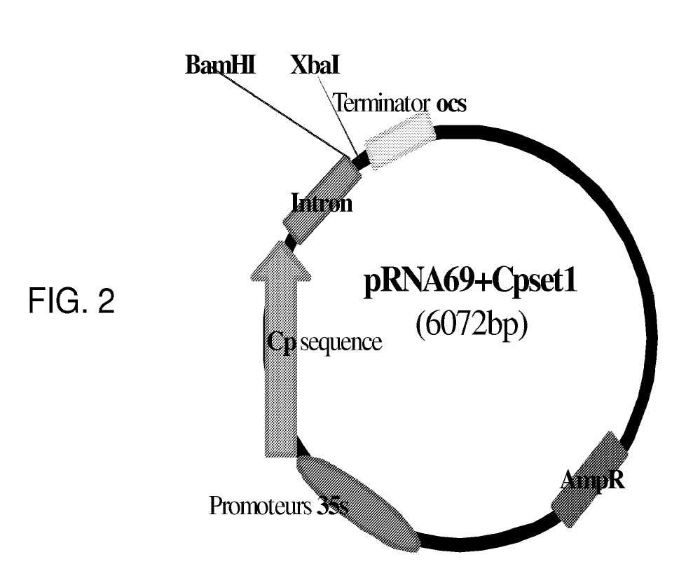

The ligated plasmids were amplified in E. coli, then selected for Ampicillin resistance. Recombinant colonies with plasmids pRNA69-CPset1 were selected, grown and plasmid DNA isolated according to manufacturer's protocol (High-Speed Plasmid Mini Kit, Genaid Biotech Ltd, Taiwan) (FIG. 2). The resulting plasmids were isolated and purified, and inserts verified by gel separation and sequencing of the fragments.

Binary Plant Expressible Plasmids for IAPV-Specific dsRNA Production

Purified pRNA69-CPset1 plasmids were double-digested with XbaI and BamI restriction enzymes, and linearized plasmid DNA gel-purified. The cloned sequence “CPset2” was released from pJET-CPset2 by digestion, purified, and ligated into the double-digested, purified pRNA69-CPset1 plasmids, according to the manufacturer's protocol (Genaid Biotech Ltd, Taiwan). The ligated plasmids were amplified in E. coli, then selected for Ampicillin resistance. Recombinant colonies with plasmids pRNA69-IR-CP were selected, grown and plasmid DNA isolated according to manufacturer's protocol (High-Speed Plasmid Mini Kit, Genaid Biotech Ltd, Taiwan). The resulting pRNA69-IR-CP plasmids bearing both “CPset1” and the inverted repeat “CPset2” inserts were isolated and purified, and inserts verified by gel separation and sequencing of the fragments.

In addition, PCR amplification of the pRNA69-IR-CP DNA with the pRNA69-specific primers further verified the insertion of the CPset2 sequence, the presence of both CPset1 and CPset2 inserts, and the insertion of the two inserts in an inverted configuration.

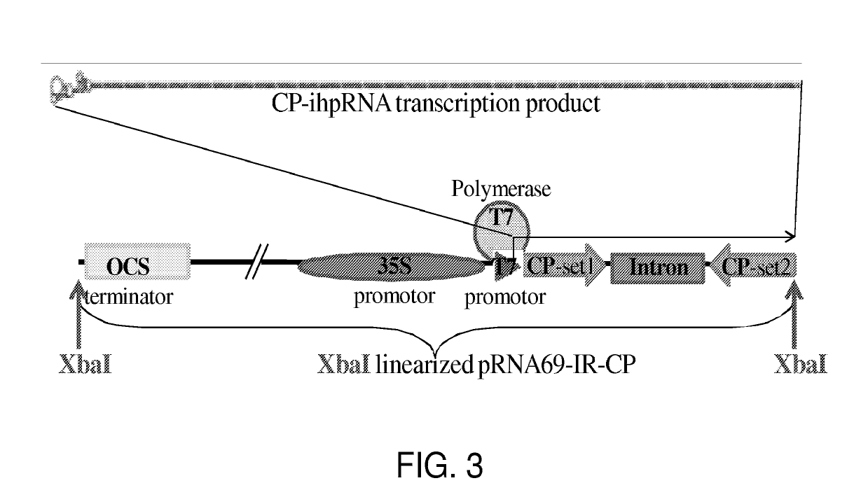

At this stage transcription from verified CP-silencing inverted-repeat constructs was performed. pRNA69-IR-CP plasmids pRNA69-IR-CP#1 and pRNA69-IR-CP#2 were linearized, ethanol precipitated, and transcription was performed with T7 polymerase followed by DICER and RNAse A digestion of the transcription products. Transcription using pRNA69 and pRNA69-CPset1 was performed as negative controls (FIG. 3).

DICER endoribonuclease RNAse III digests long dsRNA into smaller, heterogeneous siRNA fragments. RNAse A is a single strand specific RNAse, which should not cleave the long dsRNA product of pRNA69-IR-CP transcription.

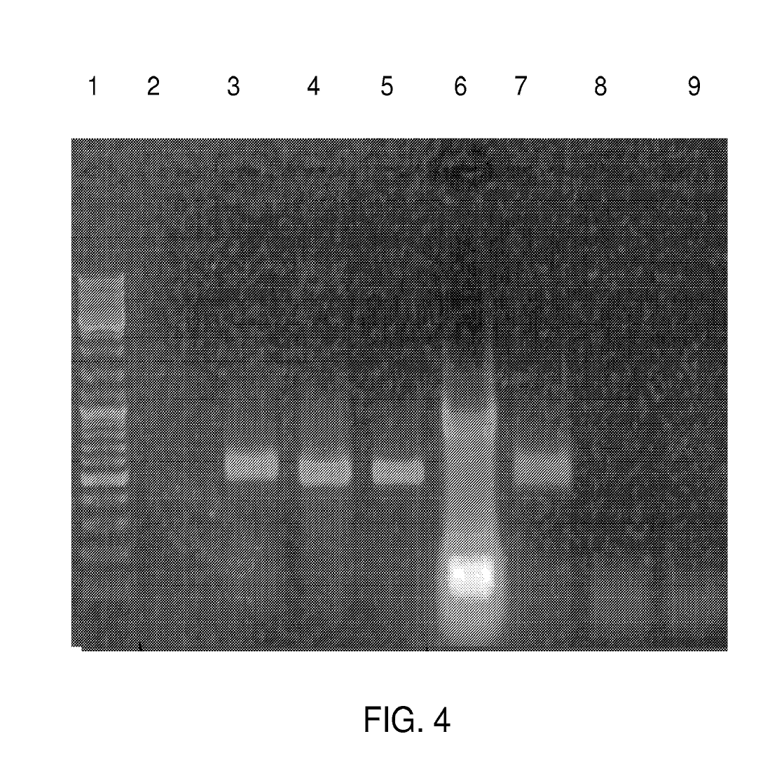

FIG. 4 shows the results of the RNAse digestions of the transcription products of pRNA69-IR-CP. The pRNA69-IR-CP transcripts (lane 3) were unaffected by RNAse A (lane 4, pRNA69-IR-CP#1, lane 5, pRNA69-IR-CP#2), although the RNAse affected complete degradation of endogenous bee single stranded RNA (lane 2). Thus, the pRNA69-IR-CP transcripts do not contain any significant amounts of single stranded RNA.

Digestion with DICER (FIG. 4, lanes 6-9), completely degraded the pRNA69-IR-CP transcripts (lane 8, pRNA69-IR-CP#1, lane 9, pRNA69-IR-CP#2), but had no effect on endogenous bee single stranded RNA (lane 6). Lane 7 shows the pRNA69-IR-CP#1 intact after incubation in the absence of DICER.

Thus, transcription of pRNA69-IR-CP plasmids, constructed as described herein, results in accumulation of accurate IAPV-specific dsRNA.

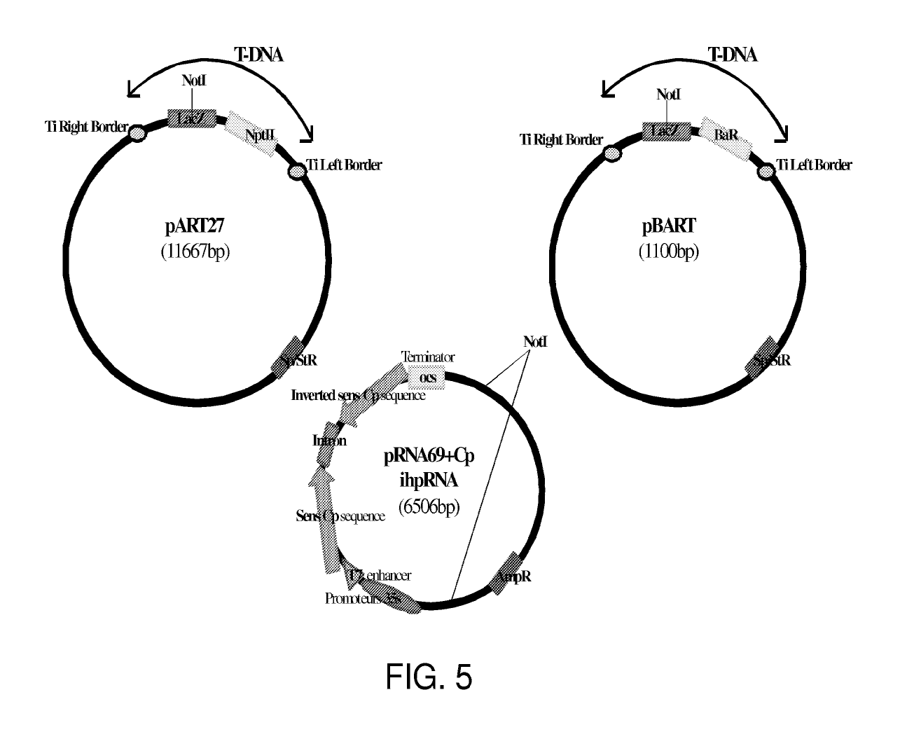

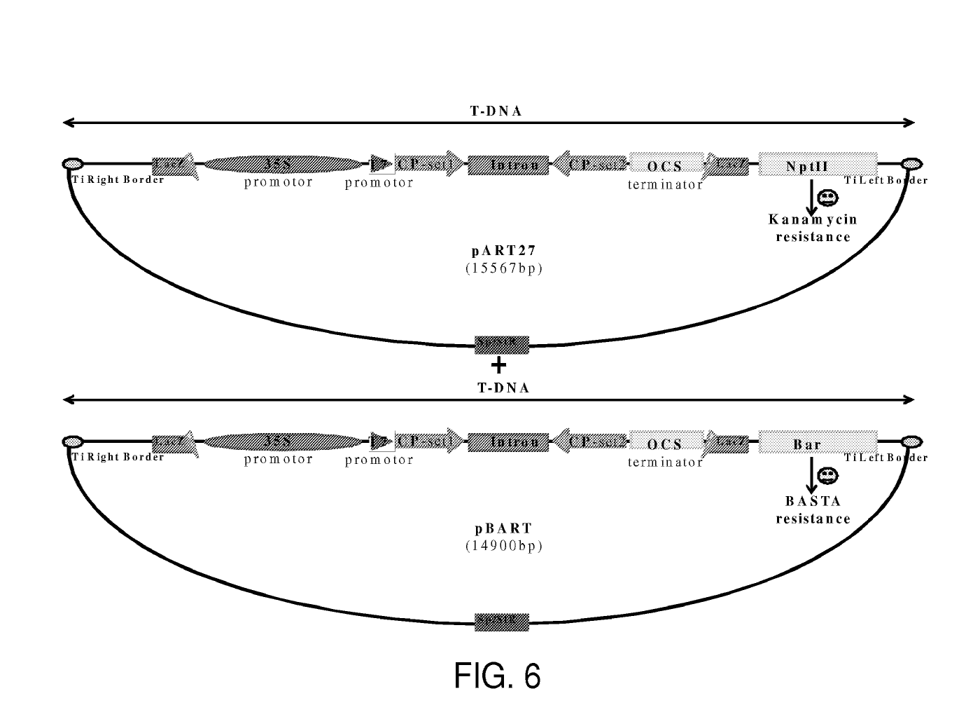

Next, the IAPV-specific cassettes of pRNA69-IR-CP (SEQ ID NO: 311533) were released from pRNA69-IR-CP by NotI digestion, and ligated into linearized binary vectors pART27 and pBART within the LacZ sequence, with no specific orientation (FIG. 5). Ligation products were transformed into E. coli and recombinant colonies selected by PCR amplification with OCS and 35S primers. Analysis of the cloned DNA revealed one clone including pBART-IR-CP #2 one orientation and another clone including pBART-IR-CP #1 in the opposite orientation (FIG. 6).

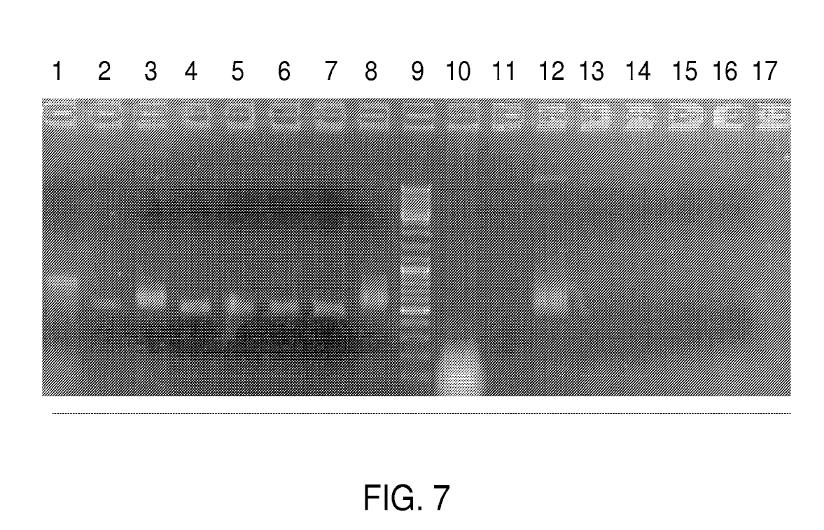

In-vitro transcription of pART27-IR-CP#1, pART27-IR-CP#2, pBART-IR-CP #1 and pBART-IR-CP#2 resulted in transcripts identical to IAPV-specific dsRNA (SEQ ID NO: 311552), as analyzed by gel electrophoresis, migrating as a 500 bp fragment (results not shown). RNAse A and DICER digestion (FIG. 7) confirmed that the transcripts were indeed dsRNA (ihpRNA), as they were completely resistant to RNAse A digestion (FIG. 7, lanes 4-7: Lane 4=pBART-IR-CP#1, lane 5=pBART-IR-CP#2, lane 6=pART27-IR-CP#1, lane 7=pART27-IR-CP#2), but were completely degraded by DICER (FIG. 7, lanes 13-16: Lane 13=pBART-IR-CP#1, lane 14=pBART-IR-CP#2, lane 15=pART27-IR-CP#1, lane 16=pART27-IR-CP#2).

Thus, the proper orientation and reading frame of the IAPV-specific insertions of the binary vectors were confirmed by restriction digestion and gel analysis, and the function of the binary vectors, and double stranded nature of the transcribed CP-ihpRNA was confirmed by the in vitro transcription assay. The binary plasmids are now ready to transform plants and plant cells via Agrobacterium-mediated transformation and integration into a plant host genome.

### Example II

### Transformation of Plants with Binary Vectors Expressing Plant Pathogen-Specific dsRNA

Generation of Transgenic Plants

Plant tissues (for example, cotton, potato, tobacco, rice, tomato, arabidopsis, rapeseed, mustard, etc) are transformed with the Agrobacterium tumefaciens strain (e.g. C58) comprising the vectors encoding plant pathogen-specific dsRNA (e.g. pBART-IR-CP#1 and pBART-IR-CP#2), and regenerated into whole plants via protocols described for example in “Transgenic plants, Methods and Protocols. (2005) Methods in Molecular Biology, Volume 286, edited by Leandro Pena, Humana Press, Totowa N.J.”. Non-limiting, exemplary transformations are detailed below by way of illustration.

A. Nicotiana tabacum Transformation

Plant transformation vectors encoding plant pathogen-specific dsRNA (e.g. pBART-IR-CP#1 and pBART-IR-CP#2, optionally with tissue, developmental or other specific promoters or enhancers) are introduced separately into Agrobacterium tumefaciens (for example, strain EHA 105) following the protocol supplied with by manufacturer [e.g. Pharmacia Agrobacterium cells (product: #27-1535)]. To prepare the Agrobacterium competent cells, a selective medium is inoculated with Agrobacterium tumefaciens and cultured, then recultured in fresh medium. The cells are harvested and resuspended in chilled CaCl₂, and frozen until use.

The competent Agrobacterium cells are transformed as follows: An aliquot (e.g. 0.5-10 μg) of uncut plasmid DNA in water are added to 100 μl Agrobacterium competent cells and incubated on ice for 30 min. The cells are then frozen in liquid nitrogen and thawed quickly, medium added and incubated for 2 h with gentle shaking (100 rpm). Cells are then harvested, the pellet resuspended, and plated on a selective medium (e.g. BASTA and kanamycin), and grown for 2-3 days. Plasmid DNA from individual Agrobacterium colonies is then digested and analyzed by agarose gel electrophoresis as described herein to verify the integrity of the vector. Individual colonies that contain the desired recombinant plasmid are selected, recultured overnight in selective medium (e.g. BASTA and kanamycin) and the cells harvested and resuspended in medium.

Agrobacterium-mediated transformation of tobacco cultivars is performed as follows. Pieces of fresh young tobacco leaves are sterilized 1-2 min in 70% ethanol, 5 min in Javex and then rinsed in sterile water for 2 min 3 times. Leaf discs are obtained with a 5 mm cork borer. Leaf discs are then transferred to a dish containing the Agrobacterium cell suspension and grown in a specific light/dark regimen for 2-3 days. The co-cultivated discs are transferred to plates with selective medium (such as TTK plates) and grown for a further 2 weeks. Regenerated shoots are transferred to vessels containing selective rooting medium (e.g. with BASTA and kanamycin). Once a good root system has developed, the plantlets are removed from the vessels, most of the agar removed from the roots and the plantlets transferred to moist potting soil.

Kanamycin resistant Nicotiana tabacum plants are demonstrated to be transformed by PCR analysis following transformation with pathogen-specific dsRNA vectors (e.g. pBART-IR-CP#1 or pBART-IR-CP#2), using specific primers, for example, T7 primers, or pBART specific primers that anneal in or near the insert regions, to amplify an insert-specific fragment. Function of the transformed binary vectors, and double stranded nature of the transcribed CP-ihpRNA is confirmed by PCR and gel analysis of transcripts in tissue extracts, as described above.

B. Brassica napus Transformation

Plant transformation vectors encoding plant pathogen-specific dsRNA (e.g. pBART-IR-CP#1 and pBART-IR-CP#2, optionally with tissue, developmental or other specific promoters or enhancers) are introduced separately into Agrobacterium tumefaciens, following the protocol supplied by the manufacturer [for example, Pharmacia Agrobacterium cells (product: #27-1535)]. To prepare the Agrobacterium competent cells, selective medium (with appropriate antibiotics, e.g. BASTA and kanamycin) is inoculated with a glycerol stock of Agrobacterium tumefaciens and cultured at by shaking at 250 rpm approximately 15 hours, an aliquot recultured to desired density, the culture chilled, cells harvested, and resusupended in cold 20 mM CaCl₂. Competent cells are then dispensed into aliquots and frozen.

The Agrobacterium cells are then transformed as follows. The competent Agrobacterium cells are transformed as follows: An aliquot (e.g. 0.5-10 μg) of uncut plasmid DNA in water are added to 100 μl Agrobacterium competent cells and incubated on ice for 30 min. The cells are then frozen in liquid nitrogen and thawed quickly, medium added and incubated for 2 h with gentle shaking (100 rpm). Cells are then harvested, the pellet resuspended, and plated on appropriate selective medium (for example, chloramphenicol, gentamycin, BASTA, kanamycin, etc.), and grown for 2-3 days. Plasmid DNA from individual Agrobacterium colonies is then digested and analyzed by agarose gel electrophoresis as described herein to verify the integrity of the vector. Individual colonies that contain the desired recombinant plasmid are selected, recultured overnight in selective medium and the cells harvested and resuspended in medium.

Agrobacterium-mediated transformation of Brassica cultivars (for example, B. napus cv. Westar) is performed according to the method of Moloney M. M., Walker, J. M., Sharma, K. K. Plant Cell Rep. 8:238-242 (1989) with modifications. Seeds are sterilized, with bleach, detergent and mercuric chloride, rinsed well with sterile distilled water and plated on ½ strength hormone-free MS medium (Sigma) with sucrose in petri dishes. They are then placed into sterilized vessels and maintained in a light/dark regimen for cotyledon growth.

Cotyledons are excised from 4-day old seedlings by gently grasping both petioles just above the point where they join the hypocotyl. The cotyledons are soaked in BASE solution (4.3 g/L MS (GIBCO BRL), 10 ml 100.times.B5 Vitamins (0.1 g/L nicotinic acid, 1.0 g/L thiamine-HCl, 0.1 g/L pyridoxine-HCl, 10 g/L m-inositol), 2% sucrose, 1 mg/L 2,4-D, pH 5.8; 1% DMSO and 200.mu.M acetosyringone added after autoclaving) containing Agrobacterium cells with the recombinant plant transformation vector. Most of the BASE solution is removed and the cotyledons incubated for 2 days in the dark. The dishes containing the cotyledons are then transferred to cold for 3-4 days in the dark, and transferred to plates containing appropriate selection medium (e.g. MS B5) and grown in a light/dark regimen until shoots appear. Shoots are transferred to vessels containing shoot selection medium, grown until desired length and transferred to vessels containing selective rooting medium (e.g. MS, B5 Vitamins, sucrose, alpha.-naphthaleneacetic acid, timentin and antibiotic, e.g. BASTA and kanamycin). Once a good root system develops, the plantlets are removed from the vessels, most of the agar removed from the roots and the plantlets transferred to moist potting soil.

Selected Brassica plants are demonstrated to be transformed by PCR analysis following transformation with pathogen-specific dsRNA vectors (e.g. pBART-IR-CP#1 or pBART-IR-CP#2), using specific primers, for example, T7 primers, or pBART specific primers that anneal in or near the insert regions, to amplify an insert-specific fragment. Function of the transformed binary vectors, and double stranded nature of the transcribed CP-ihpRNA is confirmed by PCR and gel analysis of transcripts in tissue extracts, as described above.

C. Brassica carinata Transformation

Plant transformation vectors encoding plant pathogen-specific dsRNA (e.g. pBART-IR-CP#1 and pBART-IR-CP#2, optionally with tissue, developmental or other specific promoters or enhancers) are introduced separately into Agrobacterium tumefaciens strain as described hereinabove. The Agrobacterium cells are transformed as described hereinabove, with appropriate selective media, and plasmid DNA from individual Agrobacterium colonies verified and prepared as described above.

The B. carinata cultivars' (Ethiopian mustard, e.g. B. carinata A. Braun, breeding line C90-1163) seeds are sterilized (in, for example, PPM (Plant Preservative Mixture, Plant Cell Technology Inc.)), and plated on fresh seed germination medium in petri dishes fitted inside vessels. Seeds are then incubated at for 3-4 days in an appropriate photoperiod.

Brassica carinata plants are then transformed as described by Babic, V., Datla, R. S., Scoles, G. J., Keller, W. Plant Cell Reports, 17, 183-188 (1998), optionally with modifications. Healthy green cotyledons are cut at the point where they join the hypocotyls, the petiole of each explant dipped into the Agrobacterium suspension and then transferred to petri dishes with Whatman No. 1 filter paper covering the regeneration medium (MS pH 5.8, 3% sucrose; 2 mg/L BA, 0.05 mg/L NAA and 0.7% phytagar). The explants are incubated for 2 days, then transferred to petri dishes containing appropriate selection medium (e.g. containing BASTA and kanamycin) and incubated for 2 weeks as above.

Regenerated shoots are then transferred to shoot elongation medium (MS e.g. containing gibberellic acid) in petri dishes fitted in vessels and incubated for two weeks as above. Shoots are then transferred to selective rooting media and when healthy roots appear the plantlets were transferred to soil.

Resistant Brassica plants are demonstrated to be transformed by PCR analysis following transformation with pathogen-specific dsRNA vectors (e.g. pBART-IR-CP#1 or pBART-IR-CP#2), using specific primers, for example, T7 primers, or pBART specific primers that anneal in or near the insert regions, to amplify an insert-specific fragment. Function of the transformed binary vectors, and double stranded nature of the transcribed CP-ihpRNA is confirmed by PCR and gel analysis of transcripts in tissue extracts, as described above.

D. Arabidopsis Transformation

Plant transformation vectors encoding plant pathogen-specific dsRNA (e.g. pBART-IR-CP#1 and pBART-IR-CP#2, optionally with tissue, developmental or other specific promoters or enhancers) are introduced separately into Agrobacterium tumefaciens strain as described hereinabove. The Agrobacterium cells are transformed as described hereinabove, with appropriate selective media, and plasmid DNA from individual Agrobacterium colonies verified and prepared as described above.

Arabidopsis thaliana plants are transformed using the floral dip method (Clough and Bent (1998) Plant Journal 16:735-743). Aerial parts of the plants are incubated for a few seconds in a solution containing 5% sucrose, resuspended A. tumefaciens cells from an overnight culture and 0.03% of the surfactant Silwet L-77. After inoculation plants are covered for 16 hours with a transparent plastic to maintain humidity. To increase the transformation efficiency, the procedure is repeated after one week. Watering is stopped as seeds become mature and dry seeds are harvested and cold treated for two days. After sterilization seeds are plated on an antibiotic containing growth medium for selection of transformed plants. The selected plants are transferred to soil for optimal seed production.

Establishment of Transgenic Plant Lines:

In general, T1 positive transformants are identified by selection (for example, by BASTA and kanamycin selection) and selfed to produce T2 seeds. These T2 seedlings are selected with the appropriate marker, and screened by RT-PCR for the presence of pathogen-specific dsRNA (for example, the IAPV-specific 433 bp CP sequence). The integration of the expected inverted repeat constructs in the genomic DNA of the T2 progeny is confirmed by PCR, as described above. In some embodiments, plants expressing the pathogen-specific dsRNA in a desired tissue or organ (e.g. nectary, pollen) are selected, in order to increase the probability of the insect's (e.g. honey bee) contact with the dsRNA.

The 35S promoter can be replaced by a nectar or flower specific promoter, such as the chsA (chalcone synthase) promoter from Petunia hybrida, the Linalool Synthase (LIS) promoter from Clarkia brewri and the nectary specific NEC1 promoter from Petunia hybrida, or other specific promoters as detailed hereinabove. Suggested use of such nectary and flower specific promoters has been, for example, to enhance or produce desirable components for increasing the nutritional quality of the honey that is collected from these flowers. Pollen specific promoters are described, for example, in U.S. Pat. No. 7,303,917, to Robert et al. (the Brassica tapetal oleosin-like protein promoter) and in US Patent Application 20040045053, to Greenland (the maize ZmC5 promoter).

Self-crossed lines for RNAi transgenic plants are then selected in the T3 generation by segregation analysis using the selective antibiotic markers. Nectar, and/or pollen from the transgenic plant lines, containing the pathogen-specific dsRNA, is provided in feed to the insects prior to, and during experimental infection with the pathogen(s), in order to assess the efficacy of the plant expressed dsRNA in retarding, reducing and/or preventing infection or severity thereof. When effective pollen and/or nectar-producing plants are selected and propagated, they are then are used for bee feeding trials. (Minihives experiments and field trials under controlled conditions).

### Example III

### Large-Scale Field Trials of Viral-Specific dsRNA for Prevention of IAPV-Associated Disease of Honeybees

In order to test the effectiveness of providing plants expressing insect pathogen-specific sdRNA of the present invention in the control of mortality in honey bees due to virus infection, the transgenic plants expressing IAPV-specific dsRNA are provided in the vicinity of bee hives, under field conditions, and resistance, tolerance to and mortality from IAPV infection were monitored. Paramaters monitored are higher adult bee population as measured by: visual inspection and total weight gain, which has been shown to be the best proxy to the total honeybee activity in the hive. The field trials are conducted basically as described in PCT IL2008/001440, which is incorporated herein in it's entirety by reference. PCT IL2008/001440 has disclosed that parameters of colony robustness are strongly affected by direct feeding of the IAPV-specific dsRNA.

### Materials and Methods

Small Interfering RNAs (siRNA), specific for IAPV and other known honeybee viruses are measured at least two time points—before IAPV active infection and after 14 days, to verify the presence and monitor concentration of siRNA in the treatment groups.

Visual inspection—frames of adult bees are assessed by two independent qualified and blinded inspectors for their percent coverage by adult bees. This assessment is done three times; at the outset, before placing the honey super (mid-point) and at the study conclusion, and results are compared between treated and control groups.

Total Weight Gain—weight gain from onset to midpoint and from onset to end-point. The total weight gain (weight of the full box and its full supers at the midpoint of the end point minus weight of the full box at the study onset) is measured.

Additional parameters that can be assessed to monitor the effect of the transgenic plants on basic parameters of bee colony health are numbers of capped brood, numbers of bees in the hive, returning foragers and honey production in exposed and control hives, in the absence of infection with IAPV (to assess safety) and following infection.

The bee hives from the two treatment groups are overwintered together according to regular beekeeping practices, and are then moved to two different flower patches:

1. Treatment flower patch growing a transgenic rapeseed variety expressing IAPV-specific dsRNA in its nectar.

2. Control flower patch growing either a non-transgenic rapeseed of the same variety, or a transgenic rapeseed variety expressing non-relevant dsRNA in its nectar.

At the end of the exposure both groups are infected with IAPV virus inoculums, and the hives moved together to yet another flower patch for an additional period of nectar collection.

Feeding schedule: Monthly feeding with sugar solution are given from study onset in fall (October) until Spring (March-April). When honey flow season begins all hives are supered (i.e. a honey-super box are be added to all hives with a queen excluder between the brood box and the honey super). The end point analysis is at June-July (midsummer) or at least one month after the supers are put on top of the hives.

Number and Source of animals: 72 hives, 36 hives in each treatment group. The hives of each group are marked by a different color mark on the hive.

Inclusion criteria: Prior to the commencement of the trial all hives are equalized. To equalize, all frames and bees are removed from the colonies, and are grouped by content (brood, honey/pollen, or empty frames). Frames are then randomly returned to the brood nests, and each hive body receives 5 frames of brood (at least 10% brood at least one side of the frame), 2 frames of honey (more than 50% honey on both sides of frame), and a mated queen. All queens are “sister queens”. Equilibrated hives with at least 6 adult bee's populated combs are included in the study. At time point zero (outset), hives are visually inspected by two assessors to verify equalization in terms of bee population, and the absence of clinical symptoms of disease. A hive that does not have at least 6 populated combs, or found queen-less is not included.

Exclusion criteria: Hives that are found during the study with less than 1 side of 1 comb populated with adult bees (bee's count of less than 10) are considered dead. A hive that is declared as dead according to this parameter is not be fed anymore. The dead hives are weighed and inspected formally as close as possible to the time of death.

Analytical Methods: Data collected during the study includes: frames coverage by adult bees, total weight of hive (plus super at endpoint), siRNA of relevant viruses and RT-PCR for virus detection.

Visual Inspection: Observers estimate (on a scale of zero to ten) what proportion of the side of each frame in the bottom box (and at the end point analysis also in each frame in the honey-super) is covered with bees.

The scores of the two inspectors are averaged and calculated to % coverage (score of 20 per comb=100%).

% coverage is transferred to number of bees per comb, according to the numbers set by Burgett and Burikam, 1985:

100% comb coverage at the bottom box=2,430 bees, 100% coverage of a super comb=1,570 bees.

The numbers are summarized to produce an average number of bees per hive.

Total Weight Gain: Each hive is weighed to set a starting weight. At midpoint total gross weight of the brood box is measured again, and total Weight Gain is calculated by subtracting the starting weight from the current weighing.

Each empty honey super is weighed and numbered prior to adding the honey supers to colonies. The investigator supers (add honey supers) colonies a week after the last feeding is done. At the end of the trial (at least one month later), the entire hive (deep brood supers and honey supers together) are weighed in the field. The total weight per hive at the end-point is calculated as the total gross weight as measured at end point, minus the weight of the relevant empty honey super. Then a Total Weight Gain is calculated by subtracting the starting weight from the end-point total weight calculated.

RT-PCR—Detection of Viruses (IAPV) Presence

Extraction of Nucleic Acids: Total RNA are extracted from the preserved bees using the Tri-reagent method (Sigma, St. Louis Mo., USA). Briefly, RNA are extracted by liquid phase separation and precipitation, following by re-suspention in nuclease-free water. RT-PCR: For IAPV expression analyses and for bee Actin internal controls measured amounts of RNA are subjected to one-step RT-PCR using RT-PCR System (Verso 1-Step RT-PCR Kit, Thermo scientific). RT-PCR are done with the following specific primers:

| | | Product |
|---|---|---|
| Primer name | Sequence | size |
| IAPV For (708) | 5′ AGACACCAATCACGGACCTCAC 3′ | 137 bp |
| IAPV Rev (780) | 5′ GAGATTGTTTGAGAGGGGTGG 3′ | |
| -Actin (90111) | | |
| For Bee | 5′ AGGAATGGAAGCTTGCGGTA 3′ | 181 bp |
| -Actin (90121) | 5′ AATTTTCATGGTGGATGGTGC 3′ | |
| Rev Bee | | |

Primers 708 and 780 amplify a region corresponding to nucleotides 8660-8997 in the IAPV sequence (GenBank: EF219380.1) and it are not within the IAPV-specific dsRNA target sequence.

Samples for virus presence analysis are taken at study outset and before IAPV introduction. Samples are taken from each study group. Samples consist of 10 bees randomly sampled from 6 hives in each group (60 total per treatment group). The bees are put into RNAsafe (Ambion) to preserve the RNA from degradation, or are immediately frozen.

Bee samples are kept in a −80° C. freezer until processed.

siRNA—Activity

RNA extraction: RNA is extracted using Tri-Reagent (Sigma, USA) according to protocol provided by the manufacturer. All samples treated with DNaseI and resuspended with loading buffer (90% Formamide, 0.05 Bromophenol Black, 0.05% Xylene cyanol) prior to loading on gel.

Gel electrophoresis and Blot: 10 μg of freshly prepared RNA are measured using the spectrophotometer and are loaded on 12% Acrylamide gel acrylamide:Bis acrylamide ratio) in denaturation environment (gel contains 7M Urea). After electrophoresis samples are transferred to positively charged nylon membrane using electroblotting method.

Hybridization and signal detection: In order to verify processing of dsRNA into siRNA. Membrane hybridized with freshly prepared DNA probe of IAPV segment, homolog to the IAPV-CP region. This is made using DIG PCR probe preparation Kit in DIG easyhyb solution (Roche) according to manufacturer protocol. The membrane is washed twice with 2×SSC/0.1% SDS than washed for stringency with 0.1×SSC/0.1% SDS in 65° C. Membranes are further washed using DIG Wash and Block Kit according to manufacturer protocol. Detection is performed using CSPD-star substrate. Positive control are 21nt DNA primers corresponding to the CP hybridized sequence.

Description of procedures for sample selection, preparation, and storage: Samples for virus presence analysis are taken before IAPV introduction (are marked as time 0) and 14 days after IAPV introduction date (are marked as time 14). Samples are taken from each study group. Samples will consist of 10 bees randomly sampled from 6 hives in each group (60 total per treatment group). The bees are put into RNAsafe (Ambion) to preserve the RNA from degradation. Bee samples are kept in a −80° C. freezer until processed.

Active Infection: A virus mixture that originated in bees taken from the local area where the experiment are set up are used in the study. The virus mixture is tested to verify presence of different viruses. Virus purification is according to the technique described in Maori et al. 2007a and Maori et al. 2007b. The virus concentrations are measured by a protein assay, and aliquots of the virus at the required concentration and total amount are marked and stored individually. Feeding is in sugar syrup.

### RESULTS

Content of IAPV-specific dsRNA in the transgenic plants as measured before and during the trial verifies the available sources of the dsRNA. RT-PCR measurement of quantities of the IAPV specific dsRNA in the bees further verifies effective delivery of the nucleic acid agents to the bees. Significantly greater weight gain and/or bee colony health by visual inspection of the hives provided with transgenic plants expressing the IAPV-specific dsRNA indicates an effect of the dsRNA. Correlation between the amount of the nucleic acid delivered, reduction in disease symptoms and tolerance to infection further indicates effective resistance to IAPV infection and tolerance of its effects in the infected bees.

After establishing that the transgenic plants alone did not make any difference relative to the untreated control, control colonies receiving virus without exposure to the transgenic plants, and colonies infected with the receiving virus and also exposed to the transgenic plants are compared to test the efficacy of the transgenic plants in directly preventing the IAPV symptoms. As Colony Collapse Disorder (CCD) is characterized by thinning of the affected colony due to reduced numbers of returning foragers the effect of the transgenic plants on numbers of returning foragers is monitored in the virus-infected colonies, over time.

Another important parameter characteristic of CCD is a reduction in the total number of bees in the hive, which is easily monitored in the test and control colonies, over time. Further, honey production, and flight activity data of a hive reflects not only the numbers of bees in the colony, but their overall health and robustness. Reduction in viral infection, and enhancement of overall health of the colony indicates effective delivery to the foraging bees, and dispersion of the plant-derived pathogen-specific dsRNA within the hive.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### LIST OF REFERENCES

### Additional References are Cited in the Text

Araujo R N, Santos A, Pinto F S, Gontijo N F, Lehane M J, Pereira M H. (2006) Insect Biochem Mol Biol. 36(9):683-93.
Baulcombe D. (2004). RNA silencing in plants. Nature. 431:356-63.
Carter and Thornburg (2002) The nectary-specific pattern of expression of the tobacco Nectarin I promoter is regulated by multiple promoter elements. Plant Molecular Biology 51: 451-457,
Chapin et al. (2000). Consequences of changing biodiversity. Nature. 405:234-42.
Chen and Siede (2007) Honey bee viruses. Adv Virus Res. 70:33-80.
Chen et al. (2007). Nosema ceranae is a long-present and wide-spread microsporidian infection of the European honey bee (Apis mellifera) in the United States. J Invert Path.
Cox Foster et al. (2007). A metagenomic survey of microbes in honey bee colony collapse disorder. Science._{—}18(5848):283-7
De Graaf et al. (2006). Diagnosis of American foulbrood in honey bees: a synthesis and proposed analytical protocols Lett Appl Microbiol. 43(6):583-90
Fernandez et al Plant Physiol. 2009; 151:1729-40
Gordon and Waterhouse (2006). Small RNA viruses of insects: expression in plants and RNA silencing Adv Virus Res. 68:459-502
H. T. Horner (2007) Amyloplast to chromoplast conversion in developing ornamental tobacco floral nectaries provides sugar for nectar and antioxidants for protection. American Journal of Botany. 94:12-24
Kram et al, BMC Plant Biol., 2009; 9:92
Myers N, Mittermeier R. A, Mittermeier C. G, da Fonseca G. A. B, Kent J. Biodiversity hotspots for conservation priorities. Nature. 2000; 403:853-858
Nature. 2007 Feb. 22; 445(7130):816-7
Pest Manag Sci. 2007 November; 63(11):1051-7. Incidents of bee poisoning with pesticides in the United Kingdom, 1994-2003.
Pettis et al. (2007) Am. Bee Journal 147: 595
Pimm S. L, Raven P. Biodiversity—extinction by numbers. Nature. 2000; 403:843-845
Pounds et al. (2006). Widespread amphibian extinctions from epidemic disease driven by global warming Nature; 439:161-7.
Samatarau et al. (2000). Parasitic mites of honey bees: life history, implications, and impact. Ann Rev Ent. 45:519-48
Science. 2004 Mar. 19; 303(5665):1879-81
Science. 2004 Mar. 19; 303(5665):1747.
Science. 2004 Sep. 10; 305(5690):1632-4.
Tenllado F., Diaz-Ruiz J. R. (2001). Double-stranded RNA-mediated interference with plant virus infection. Journal of Virology 75: 12288-12297.
Tenllado F, Llave C, Diaz-Ruiz J R. (2004). RNA interference as a new biotechnological tool for the control of virus diseases in plants. Virus Res. 102:85-96.
Timmons L, Fire A. (1998) Specific interference by ingested dsRNA. Nature. 29; 395(6705):854.
Timmons L., Court D. L., Fire A. (2001). Ingestion of bacterially expressed dsRNAs can produce specific and potent genetic interference in Caenorhabditis elegans. Gene 263:103-112.
Trends Biotechnol. 2005 August; 23(8):388-94. Effects of biotechnology on biodiversity: herbicide-tolerant and insect-resistant GM crops
Turner C T, Davy M W, MacDiarmid R M, Plummer K M, Birch N P, Newcomb R D (2006) RNA interference in the light brown apple moth, Epiphyas postvittana (Walker) induced by double-stranded RNA feeding. Insect Mol Biol. June; 15(3):383-91.
WO/2003/004649, method of interfering with a virus infection in plants.

What is claimed is:

1. A nucleic acid construct comprising a polynucleotide expressing a nucleic acid agent downregulating expression of a gene product of an insect pathogen, said polynucleotide operably linked to a cis-regulatory element operable in plants.

2. The nucleic acid construct of claim 1, wherein said nucleic acid agent is selected from the group consisting of a dsRNA, an antisense RNA and a ribozyme.

3. The nucleic acid construct of claim 2, wherein said dsRNA is selected from the group consisting of siRNA, shRNA and miRNA.

4. The nucleic acid construct of claim 1, said cis regulatory element being active in floral nectary tissues, extrafloral nectarines and/or pollen.

5. The nucleic acid construct of claim 1, wherein said nucleic acid agent is greater than 15 base pairs in length.

6. The nucleic acid construct of claim 5, wherein said nucleic acid agent is 19 to 25 base pairs in length.

7. The nucleic acid construct of claim 5, wherein said nucleic acid agent is greater than 30 base pairs in length.

8. The nucleic acid construct of claim 1, wherein said insect is a bee.

9. The nucleic acid construct of claim 1, wherein said insect pathogen is selected from the group consisting of a virus, a bacteria, a parasitic protozoan, a fungus a nematode and a mite.

10. The nucleic acid construct of claim 1, wherein said insect pathogen is a virus.

11. The nucleic acid construct of claim 10, wherein said insect is a bee and said virus is Israel Acute Paralysis Virus.

12. The nucleic acid construct of claim 11, wherein the nucleic acid agent is complementary to a viral nucleic acid sequence as set forth in any of SEQ ID NOs: 256752, 256761, 256762 and 311552.

13. The nucleic acid construct of claim 11, wherein said nucleic acid agent is complementary to a viral nucleic acid sequence as set forth in any of SEQ ID NOs: SEQ ID NOs: 256782-266260.

14. The nucleic acid construct of claim 11, having a nucleic acid sequence as set forth in SEQ ID NO: 311533.

15. The nucleic acid construct of claim 1, wherein said insect is a bee and said pathogen is a Nosema parasite.

16. The nucleic acid construct of claim 15, wherein said nucleic acid agent is complementary to a Nosema nucleic acid sequence as set forth in any of SEQ ID NOs: 55-252698.

17. The nucleic acid construct of claim 15, wherein said Nosema parasite is N. cerana and said gene product is an mRNA encoding a Nosema ATP/ADP transporter protein or homologue thereof.

18. The nucleic acid construct of claim 17, wherein said ATP/ADP transporter protein or homologue thereof is selected from the group consisting of proteins encoded by SEQ ID NOs: 44, 45, 46 and 47.

19. The nucleic acid construct of claim 1, wherein said insect is a bee and said pathogen is a Varroa destructor mite.

20. The nucleic acid construct of claim 19, wherein said gene product is an Varroa specific mRNA encoding a polypeptide selected from the group consisting of NADH dehydrogenase subunit 2, ATP synthetase subunit 8, ATP synthetase subunit 6, sodium channel and cytochrome oxydase subunit I.

21. The nucleic acid construct of claim 1, wherein said insect is a bee and said nucleic acid construct comprises at least two nucleic acid agents, each of said nucleic acid agents downregulating expression of at least one gene product of at least one bee pathogen.

22. The nucleic acid construct of claim 21, wherein said insect is a bee and said at least one bee pathogen is selected from the group consisting of a virus, a bacteria, a parasitic protozoan, a fungus, a nematode and a mite.

23. The nucleic acid construct of claim 22, wherein said at least one bee pathogen comprises at least two bee pathogens.

24. The nucleic acid construct of claim 23, wherein said nucleic acid agent corresponds to conserved sequences in said at least two bee pathogens.

25. A cell comprising the nucleic acid construct of any of claims 1-24.

26. The cell of claim 25, being a plant cell.

27. The cell of claim 25, wherein said cell is an Agrobacterium.

28. A plant expressing the nucleic acid construct of any of claims 1 to 24.

29. The plant of claim 28, wherein said plant is a flowering plant selected from the group consisting of rapeseed, alfalfa and clover.

30. The plant of claim 28, expressing said nucleic acid construct in floral nectary tissues, extrafloral nectarines or pollen.

31. A method of reducing the susceptibility of a insect to a disease caused by an insect pathogen, the method comprising providing to the insects plants expressing the nucleic acid construct of any of claims 1 to 24, such that said insect feeds from said plants and ingests said nucleic acid agent, thereby reducing the susceptibility of said insect to said pathogen.

32. The method of claim 31, wherein said insect is a pollinating insect or a herbivorous insect.

33. A method for reducing the susceptibility of a bee to a disease caused by an insect pathogen comprising providing the bee with a plant expressing the nucleotide construct of any of claims 11 to 20 such that said bee feeds from said plants and ingests said nucleic acid agent, thereby reducing the susceptibility of said bee to said pathogen.

34. The method of claim 33, wherein said insect is a bee and said transgenic plants express a nucleic acid agent complementary to a nucleic acid sequence selected from the group consisting of SEQ ID NOs: 60-252698, 252699-256728 and 256782-266260.

35. The method of claim 31, wherein said plants express a nucleic acid agent complementary to a nucleic acid sequence selected from the group consisting of SEQ ID NOs: 55-59, 253194, 254090, 259289, 256752, 256761, 256762 and 311552.

36. A method of reducing the susceptibility of honeybees to Colony Collapse Disorder (CCD), the method comprising providing to the honeybees plants expressing the nucleic acid construct of any of claims 11 to 20.

37. A method of producing a plant capable of reducing the susceptibility of an insect feeding from said plant, plant tissues or plant secretions to a disease caused by an insect pathogen, the method comprising transforming cells of said plant with the nucleic acid construct of any of claims 1 to 24, selecting transformed plants or plant cells expressing insect pathogen-specific dsRNA, and propagating said plants or plant cells to produce a plant or plant cells capable of reducing the susceptibility of an insect feeding from said plant.
